SEQUENCE LISTING (13 sequences)

SEQ ID NO: 1 - DNA, 29 bp, Homo sapiens
ccggtcttttgggggtggtgggtgaccgg

SEQ ID NO: 2 - DNA, 32 bp, Homo sapiens
ccggtcagtgggagtggtcttccattgaccgg

SEQ ID NO: 3 - DNA, 32 bp, Homo sapiens
ccggtccctgttgtgttgtttggtaagaccgg

SEQ ID NO: 4 - DNA, 33 bp, Homo sapiens
ccggtccttctatgctgttttttgtttgaccgg

SEQ ID NO: 5 - DNA, 32 bp, Homo sapiens
ccggtctggtcatctttaaagtttttgaccgg

SEQ ID NO: 6 - DNA, 32 bp, Homo sapiens
ccggtcagggttagtatggatttcttgaccgg

SEQ ID NO: 7 - DNA, 32 bp, Homo sapiens
ccggtctctctcaaaacccactcatggaccgg

SEQ ID NO: 8 - DNA, 32 bp, Homo sapiens
ccggtcttgtgcgcatgtagaatctggaccgg

SEQ ID NO: 9 - DNA, 30 bp, Artificial Sequence
  GAPDH Beacon
cgacgggagtccttccacgataccacgtcg

SEQ ID NO: 10 - DNA, 35 bp, Artificial sequence
  ALPL BEACON
cgctccagagtgtcttccgaggaggtcaaggagcg

SEQ ID NO: 11 - DNA, 40 bp, Artificial sequence
  COL1A1 BEACON
cgtcccaaaaaaaaaaaaaaaaagaaaaatatcagggacg

SEQ ID NO: 12 - DNA, 38 bp, Artificial sequence
  BGLAP BEACON
tccgccggaaagaagggtgcctggagaggagcggcgga

SEQ ID NO: 13 - DNA, 35 bp, Artificial sequence; identical to SEQ ID NO: 10
  Beacon

(19) United States
(12) Patent Application Publication
(10) Pub. No.: US 2015/0140558 A1
(43) Pub. Date: May 21, 2015

(54) Methods of Identifying and Isolating Cells Using Molecular Beacons

(71) Applicant: Brown University, Providence, RI (US)

(72) Inventors: Eric M. DARLING, Cumberland, RI (US); Hetal D. MARBLE, Providence, RI (US)

(21) Appl. No.: 14/478,272

(22) Filed: Sep. 5, 2014

Related U.S. Application Data
(60) Provisional application No. 61/874,070, filed on Sep. 5, 2013.

(51) Int. Cl. C12N 5/071

(52) U.S. Cl. CPC C12N 5/0602
USPC 435/6.11; 435/377

(57) ABSTRACT

Methods are provided for isolating lineage-specific cells from a population of cells using molecular beacons which bind to mRNA markers and then isolating the lineage specific cells from the population of cells based on the label of the molecular beacon.

### RELATED APPLICATION

This application claims priority to U.S. Provisional Patent Application No. 61/874,070, filed on Sep. 5, 2013 and is hereby incorporated herein by reference in its entirety for all purposes.

### STATEMENT OF GOVERNMENT INTERESTS

This invention was made with government support under AR054673 and GM104937 awarded by the National Institutes of Health. The government has certain rights in the invention.

### BACKGROUND

1. Field of the Invention

Embodiments of the present invention relate in general to methods and compositions for identifying cells from a population of cells, such as lineage-specific cells, using molecular beacons and to methods of designing molecular beacons.

2. Description of Related Art

Molecular beacons and their use to detect live cells is generally known in the art. See Tyagi et al., Molecular Beacons: Probes That Fluoresce Upon Hybridization, Nature Biotechnology, 14:303-308 (1996); Rhee et al., Target Accessibility and signal specificity in Live-Cell Detection of BMP-4 mRNA Using Molecular Beacons, Nucleic Acids Res, 36(5):e30 (2008); King et al., High-Throughput Tracking of Pluripotent Human Embryonic Stem Cells with Dual Fluorescence Resonance Energy Transfer Molecular Beacons, Stem Cells Dev. 20:475-84 (2011); Larsson et al., Sorting Live Stem Cells Based on Sox2 mRNA Expression, PLoSOne 7(11):349874 (2012); and Lenaerts et al., Improved Fluorescent In Situ Hybridization Method for Detection of Bacteria from Activated Sludge and River Water by Using DNA Molecular Beacons and Flow Cytometry, Appl. Environ. Microbiol. 73(6):2020-3 (2007) each of which are hereby incorporated by reference in their entireties.

### SUMMARY

The present invention provides methods and compositions to identify and/or isolate cells using molecular beacons to bind to a mRNA marker indicative of the cell. Cells can be lineage-specific cells or they can be malignant cells or any cell where the object is to identify the presence of the cell or otherwise isolate the cell from a population of cells.

According to one aspect, a method is provided for enriching live tissue lineage-specific cells from a population of live cells including subjecting the population of live cells to conditions inducing differentiation to a specific tissue type, wherein differentiation is induced in a plurality of the live cells to produce live tissue lineage-specific cells expressing a mRNA marker, hybridizing the mRNA marker within the live tissue lineage-specific cells to a labeled oligonucleotide, such as a molecular beacon having a label attached thereto, and isolating the live tissue lineage-specific cells based on the label. It is to be understood that reference to a molecular beacon herein may also refer to a more general labeled oligonucleotide for hybridizing to the mRNA marker. According to certain aspects, the live cells include unipotent, bipoptent, multipotent, pluripotent or totipotent cells. According to certain aspects, the live cells include stem cells, embryonic stem cells, pluripotent stem cells, induced pluripotent stem cells, mesenchymal stem cells, somatic adult stem cells, stromal cells or progenitor cells. According to certain aspects, the label is a fluorescent label. According to certain aspects, the label is a fluorescent label and the live tissue lineage-specific cells are isolated based on the fluorescent label. According to certain aspects, the label is a fluorescent label and the live tissue lineage-specific cells are isolated using fluorescence activated cell sorting. According to certain aspects, the label is a magnetic label. According to certain aspects, the label is a magnetic label and the live tissue lineage-specific cells are isolated based on the magnetic label. According to certain aspects, the label is a magnetic label and the live tissue lineage-specific cells are isolated using a magnetic field. Such methods based on a magnetic field include magnetic-activated cell sorting (MACS), magnetic resonance imaging (MRI) and nuclear magnetic resonance (NMR). According to certain aspects, MACS has particular application to sorting cells and MRI and NMR have particular application to diagnostic approaches described herein.

According to one aspect, a method is provided for identifying live tissue lineage-specific cells within a population of live cells including subjecting the population of live cells to conditions inducing differentiation to a specific tissue type, wherein differentiation is induced in a plurality of the live cells to produce live tissue lineage-specific cells expressing a mRNA marker, hybridizing the mRNA marker within the live tissue lineage-specific cells to a molecular beacon having a label attached thereto, and identifying the live tissue lineage-specific cells based on the label. According to certain aspects, the live cells include unipotent, bipoptent, multipotent, pluripotent or totipotent cells. According to certain aspects, the live cells include stem cells, embryonic stem cells, pluripotent stem cells, induced pluripotent stem cells, mesenchymal stem cells, somatic adult stem cells, stromal cells or progenitor cells. According to certain aspects, the label is a fluorescent label. According to certain aspects, the label is a fluorescent label and the live tissue lineage-specific cells are isolated based on the fluorescent label. According to certain aspects, the label is a fluorescent label and the live tissue lineage-specific cells are isolated using fluorescence activated cell sorting. According to certain aspects, the label is a magnetic label. According to certain aspects, the label is a magnetic label and the live tissue lineage-specific cells are isolated based on the magnetic label. According to certain aspects, the label is a magnetic label and the live tissue lineage-specific cells are isolated using a magnetic field. Such methods based on a magnetic field include magnetic-activated cell sorting (MACS), magnetic resonance imaging (MRI) and nuclear magnetic resonance (NMR). According to certain aspects, MACS has particular application to sorting cells and MRI and NMR have particular application to diagnostic approaches described herein.

According to one aspect, a method is provided of optimizing production of a specific tissue type from cells induced to differentiate into the specific tissue type including subjecting a population of live cells to conditions inducing differentiation to the specific tissue type, wherein differentiation is induced in a plurality of the live cells to produce live tissue lineage-specific cells expressing a mRNA marker, hybridizing the mRNA marker within the live tissue lineage-specific cells to a molecular beacon having a label attached thereto, isolating the live tissue lineage-specific cells based on the label, and further inducing differentiation of the isolated live tissue lineage-specific cells into the specific tissue type, i.e. to produce neotissue. According to certain aspects, the live cells include unipotent, bipoptent, multipotent, pluripotent or totipotent cells. According to certain aspects, the live cells include stem cells, embryonic stem cells, pluripotent stem cells, induced pluripotent stem cells, mesenchymal stem cells, somatic adult stem cells, stromal cells or progenitor cells. According to certain aspects, the label is a fluorescent label. According to certain aspects, the label is a fluorescent label and the live tissue lineage-specific cells are isolated based on the fluorescent label. According to certain aspects, the label is a fluorescent label and the live tissue lineage-specific cells are isolated using fluorescence activated cell sorting. According to certain aspects, the label is a magnetic label. According to certain aspects, the label is a magnetic label and the live tissue lineage-specific cells are isolated based on the magnetic label. According to certain aspects, the label is a magnetic label and the live tissue lineage-specific cells are isolated using a magnetic field. Such methods based on a magnetic field include magnetic-activated cell sorting (MACS), magnetic resonance imaging (MRI) and nuclear magnetic resonance (NMR). According to certain aspects, MACS has particular application to sorting cells and MRI and NMR have particular application to diagnostic approaches described herein.

According to one aspect, a method is provided for identifying a malignant cell having a target mRNA marker including hybridizing the mRNA marker within the malignant cell to a molecular beacon having a label attached thereto, and identifying the malignant cell based on the label. According to one aspect, the malignant cell is a cancer cell. According to certain aspects, the label is a fluorescent label. According to certain aspects, the label is a fluorescent label and the malignant cells are isolated based on the fluorescent label. According to certain aspects, the label is a fluorescent label and the malignant cells are isolated using fluorescence activated cell sorting. According to certain aspects, the label is a magnetic label. According to certain aspects, the label is a magnetic label and the malignant cells are isolated based on the magnetic label. According to certain aspects, the label is a magnetic label and the malignant cells are isolated using a magnetic field. Such methods based on a magnetic field include magnetic-activated cell sorting (MACS), magnetic resonance imaging (MRI) and nuclear magnetic resonance (NMR). According to certain aspects, MACS has particular application to sorting cells and MRI and NMR have particular application to diagnostic approaches described herein.

According to one aspect, a method is provided for identifying a malignant cell having a target mRNA marker within a population of cells including hybridizing the mRNA marker within the malignant cell to a molecular beacon having a label attached thereto, and identifying the malignant cell within the population of cells based on the label. According to one aspect, the malignant cell is a cancer cell. According to certain aspects, the label is a fluorescent label. According to certain aspects, the label is a fluorescent label and the malignant cells are isolated based on the fluorescent label. According to certain aspects, the label is a fluorescent label and the malignant cells are isolated using fluorescence activated cell sorting. According to certain aspects, the label is a magnetic label. According to certain aspects, the label is a magnetic label and the malignant cells are isolated based on the magnetic label. According to certain aspects, the label is a magnetic label and the malignant cells are isolated using a magnetic field. Such methods based on a magnetic field include magnetic-activated cell sorting (MACS), magnetic resonance imaging (MRI) and nuclear magnetic resonance (NMR). According to certain aspects, MACS has particular application to sorting cells and MRI and NMR have particular application to diagnostic approaches described herein.

According to one aspect, a method is provided for enriching live tissue lineage-specific cells from a population of live cells wherein the live tissue lineage-specific cells express a mRNA marker including hybridizing the mRNA marker within the live tissue lineage-specific cells to a molecular beacon having a label attached thereto, and isolating the live tissue lineage-specific cells based on the label. According to certain aspects, the live cells include unipotent, bipoptent, multipotent, pluripotent or totipotent cells. According to certain aspects, the live cells include stem cells, embryonic stem cells, pluripotent stem cells, induced pluripotent stem cells, mesenchymal stem cells, somatic adult stem cells, stromal cells or progenitor cells. According to certain aspects, the label is a fluorescent label. According to certain aspects, the label is a fluorescent label and the live tissue lineage-specific cells are isolated based on the fluorescent label. According to certain aspects, the label is a fluorescent label and the live tissue lineage-specific cells are isolated using fluorescence activated cell sorting. According to certain aspects, the label is a magnetic label. According to certain aspects, the label is a magnetic label and the live tissue lineage-specific cells are isolated based on the magnetic label. According to certain aspects, the label is a magnetic label and the live tissue lineage-specific cells are isolated using a magnetic field. Such methods based on a magnetic field include magnetic-activated cell sorting (MACS), magnetic resonance imaging (MRI) and nuclear magnetic resonance (NMR). According to certain aspects, MACS has particular application to sorting cells and MRI and NMR have particular application to diagnostic approaches described herein.

According to one aspect, a method is provided for identifying live tissue lineage-specific cells within a population of live cells wherein the live tissue lineage-specific cells express a mRNA marker including hybridizing the mRNA marker within the live tissue lineage-specific cells to a molecular beacon having a label attached thereto, and identifying the live tissue lineage-specific cells based on the label. According to certain aspects, the live cells include unipotent, bipoptent, multipotent, pluripotent or totipotent cells. According to certain aspects, the live cells include stem cells, embryonic stem cells, pluripotent stem cells, induced pluripotent stem cells, mesenchymal stem cells, somatic adult stem cells, stromal cells or progenitor cells. According to certain aspects, the label is a fluorescent label. According to certain aspects, the label is a fluorescent label and the live tissue lineage-specific cells are isolated based on the fluorescent label. According to certain aspects, the label is a fluorescent label and the live tissue lineage-specific cells are isolated using fluorescence activated cell sorting. According to certain aspects, the label is a magnetic label. According to certain aspects, the label is a magnetic label and the live tissue lineage-specific cells are isolated based on the magnetic label. According to certain aspects, the label is a magnetic label and the live tissue lineage-specific cells are isolated using a magnetic field. Such methods based on a magnetic field include magnetic-activated cell sorting (MACS), magnetic resonance imaging (MRI) and nuclear magnetic resonance (NMR). According to certain aspects, MACS has particular application to sorting cells and MRI and NMR have particular application to diagnostic approaches described herein.

Further features and advantages of certain embodiments of the present invention will become more fully apparent in the following description of the embodiments and drawings thereof, and from the claims.

### BRIEF DESCRIPTION OF THE DRAWINGS

FIG. 1A is a schematic representation of cells having positive signals for specific genes of interest. FIG. 1B is an image showing fluorescence signal.

FIG. 2 shows images of fluorescent signals.

FIG. 3 is a graph depicting percentage of cells expressing ALPL, COL1A1 and BGLAP measured daily from days 2-12 and fluorescence images.

FIG. 4 is a graph of alkaline phosphatase activity.

FIG. 5A is a graph of calcium phosphate deposition. FIG. 5B is an image of a calcified matrix deposition in induced cells. FIG. 5C is an image of control cells.

FIG. 6 is a graph of alkaline phosphatase molecular beacon activity.

FIG. 7 is a graph of osteogenic beacon persistence in MG-63 cells.

FIG. 8A is a graph of gene expression patterns for ALPL. FIG. 8B is a graph of gene expression patterns for COL1A1. Figure S3C is a graph of gene expression patterns for BGLAP.

FIG. 9A is a graph of relative COL1A1 mRNA levels. FIG. 9B is a graph of relative BGLAP mRNA levels.

FIG. 10A is a graph of sort data showing distribution of fluorescence intensity in P0, single-donor ASCs with ASCs treated with ALPL molecular beacon 4 days after osteogenic induction. 48% of induced cells are ALPL+, while 46% of induced cells are ALPL−. 95% of the non-induced cells are ALPL−. FIG. 10B shows data wherein ASCs stained with antibodies for CD31 and CD34. 39% of cells display the desired phenotype of CD34+/CD31− (EC, endothelial cells; ASC, adipose-derived stromal cells).

FIG. 11 is a graph and images quantifying calcified matrix deposited by beacon-sorted ASCs by measuring the absorbance at 540 nm of the eluted Alizarin Red-S dye (mean value±std. dev.). ALPL+ cells display a 6- and 7-fold increase in matrix deposition over unsorted and ALPL− cells, respectively (all unmatched letters are significant with p<0.05 determined with two factor ANOVA and SNK post-hoc analysis using log transformed data).

FIG. 12 is a graph and images quantifying lipid produced by beacon-sorted ASCs by measuring the absorbance at 500 nm of the eluted dye Oil Red 0 (mean value±std. dev.). ALPL+ cells display a 1.6-fold increase in matrix deposition over unsorted and ALPL− cells (asterisks are significant with p<0.05 determined with two factor ANOVA and Fisher's LSD post-hoc analysis).

FIG. 13 is a graph quantifying chondrogenic differentiation of beacon-sorted ASCs by measuring the absorbance at 525 nm of dimethylmethylene blue dye binding to secreted sulfated glycosaminoglycans (mean value±std. dev.). ALPL+ cells display a 1.7- and 0.9-fold increase in matrix deposition over unsorted and ALPL− cells (all unmatched letters are significant with p<0.05 determined by two factor ANOVA and Fisher's LSD post-hoc analysis).

### DETAILED DESCRIPTION

Terms and symbols of nucleic acid chemistry, biochemistry, genetics, and molecular biology used herein follow those of standard treatises and texts in the field, e.g., Komberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); and the like.

The present invention provides methods and compositions and molecular beacons for identifying, measuring and quantifying temporal gene expression patterns in living cells undergoing differentiation. Molecular beacons include a label, such as a fluorescent label, and a quencher. When in a quenched state, the label is not detectable. When the molecular beacon hybridizes to its target, the label and the quencher are separated and the label is activated. Although, molecular beacons generally may include fluorescent labels, other labels may be used as described herein using methods known to those of skill in the art. According to one aspect, the methods described herein allow determination of heterogeneity in differentiating cell cultures. According to another aspect, the methods described herein allow measuring the percentage of positively signaling cells to show dynamic expression patterns of differentiating cells. According to one aspect, molecular beacons described herein are taken into cells in a population. The molecular beacons hybridize to mRNA markers within the cells. Once hybridized to the mRNA markers, the label of the molecular beacon is activated as is known in the art. The cell with the detectable label therein may then be detected and/or identified.

According to one aspect, methods are provided for determining gene expression in a population of differentiating cells using molecular beacons as described above which target particular mRNA markers of differentiation. Using detection and measurement of the label, and therefore expression of the target gene, patterns of gene regulation, such as upregulation and downregulation, can be monitored as a function of time. Peak gene expression times for differentiating cells can be identified. In this manner, methods described herein provide a temporal analysis of gene expression in differentiating cells from initial induction of differentiation to final tissue type. In this manner, methods described herein allow the determination of the effect of certain conditions and/or reagents on cell differentiation. In this manner, the use of molecular beacons in the methods described herein allow the quantification of the rate of differentiation of cells while taking into account the heterogeneity in the sample of differentiating cells.

According to one aspect, methods are provided to identify dynamic gene expression patterns for characteristic genes in differentiation. According to one aspect, methods are provided by which gene expression can be assessed in differentiating live-cells over extended periods.

According to certain aspects, methods of designing molecular beacons are provided for targeting genes characteristic of cell lineages, by hybridizing to corresponding mRNA markers, and are used to generate gene expression timelines. Additionally, stem cell populations from diverse sources could be monitored in this way to compare their heterogeneity and differentiation capability. Further methods are provided for using molecular beacons to determine the effectiveness of differentiation media or stimulation techniques in live-cell, experimental designs.

According to certain aspects, methods of the present invention are directed to isolating or separating cells undergoing differentiation from a population of cells which has been subjected to conditions capable of inducing differentiation.

Cells according to the present disclosure include cells which are capable of differentiation. Such cells include unipotent cells, bipotent cells, multipotent cells, pluripotent cells and totipotent cells. One of skill will readily understand and be able to identify exemplary unipotent cells, bipotent cells, multipotent cells, pluripotent cells and totipotent cells based on the present disclosure. Cells according to the present invention include stem cells, mesenchymal stem cells (including adipose-derived stem cells), progenitor cells, and mature, terminally differentiated celltypes that are capable of transdifferentiation including but not limited to fibroblasts, endothelial cells and smooth muscle cells. One of skill will readily understand and be able to identify exemplary stem cells, mesenchymal stem cells (including adipose-derived stem cells), progenitor cells, and mature, terminally differentiated celltypes that are capable of transdifferentiation based on the present disclosure.

Such cell types may be induced to differentiate into desired tissue types. Such induced cells are referred to as lineage-specific cells insofar as they have been subjected to conditions which begin differentiation of the cells into specific tissue types. Useful tissue types or lineage specific cells include adipogenic cells, neurogenic cells, osteogenic cells, chondrogenic cells and peripheral blood mononuclear cells, myogenic cells, hepatogenic cells, alveolar lung cells, pancreatic cells and kidney cells. One of skill will readily understand and be able to identify exemplary lineage-specific cells and/or tissue types based on the present disclosure.

Differentiation conditions are well known to those of skill in the art to induce differentiation of cells into lineage-specific cells. Exemplary differentiation conditions are described in Bunnell et al., Differentiation of adipose stem cells, Methods Mol. Biol., 456: 155-71 (2008); Cheng et al., Chondrogenic differentiation of adipose-derived adult stem cells by a porous scaffold derived from native articular cartilage extracellular matrix, Tissue Engineering Part A, 15:231-41 (2009) and Guilak et al., Clonal analysis of the differentiation potential of human adipose-derived adult stem cells, J. Cell Physiol. 206:229-37 (2006) each of which are hereby incorporated by reference in its entirety. One of skill will readily be able to identify additional conditions for cell differentiation based on the present disclosure.

Markers for different cell lineages include mRNA corresponding to genes characteristically expressed during cell differentiation. For example, mRNA molecules coding for alkaline phosphatase, type I collagen and osteocalcin (ALPL, COL1A1 and BGLAP) are exemplary target mRNA markers for genes characteristically expressed during osteogenesis. It is to be understood that one of skill in the art can readily identify mRNA markers for lineage-specific cells, malignant cells or any desired cell. Once the known mRNA marker is selected, one or more molecular beacons may be designed and used as described herein.

Molecular beacons according to the present disclosure include those known to exist to those of skill in the art and in the literature. Molecular beacons are hairpin-shaped nucleic acid probes functionalized with a label, such as a fluorophore, and a quencher on opposing ends. See Tyagi S, Kramer F R., Molecular beacons: probes that fluoresce upon hybridization, Nature Biotechnology, 14:303-8 (1996) hereby incorporated by reference in its entirety. The loop region of the probe is complimentary to a nucleic acid sequence of interest. In the absence of the target sequence, the probe retains its stem-loop structure and fluorescence is quenched. When the target sequence is bound by the loop region, the stem unfolds, affording fluorescence. Molecular beacons have been used in many capacities, including single nucleotide polymorphism (SNP) detection, real-time PCR applications, and many live cell imaging applications. See Manganelli R, Tyagi S, Smith I., Real Time PCR Using Molecular Beacons: A New Tool to Identify Point Mutations and to Analyze Gene Expression in Mycobacterium tuberculosis, Methods in Molecular Medicine. 54:295-310 (2001), Mhlanga M M, Vargas D Y, Fung C W, Kramer F R, Tyagi S., tRNA-linked molecular beacons for imaging mRNAs in the cytoplasm of living cells, Nucleic Acids Research, 33:1902-12 (2005), Rhee W J, Bao G., Simultaneous detection of mRNA and protein stem cell markers in live cells, BMC Biotechnology, 9:30 (2009), Santangelo P J, Nix B, Tsourkas A, Bao G., Dual FRET molecular beacons for mRNA detection in living cells, Nucleic Acids Research, 32:e57 (2004), Tsourkas A, Bao G., Shedding light on health and disease using molecular beacons, Briefings in Functional Genomics & Proteomics, 1:372-84 (2003), and Baker M B, Bao G, Searles C D., In vitro quantification of specific microRNA using molecular beacons, Nucleic Acids Research, 40:e13 (2012) each of which is hereby incorporated by reference in its entirety.

Visually detectable markers suitable for use in the molecular beacons described herein may be positively and negatively selected and/or screened using technologies such as fluorescence activated cell sorting (FACS) or microfluidics. Examples of detectable markers include various enzymes, prosthetic groups, fluorescent markers, luminescent markers, bioluminescent markers, and the like. Examples of suitable fluorescent moieties include, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, dansyl chloride, phycoerythrin and the like. Examples of suitable bioluminescent markers include, but are not limited to, luciferase (e.g., bacterial, firefly, click beetle and the like), luciferin, aequorin and the like. Examples of suitable enzyme systems having visually detectable signals include, but are not limited to, galactosidases, glucorinidases, phosphatases, peroxidases, cholinesterases and the like. Other suitable markers useful for molecular beacon design are known to those of skill in the art.

According to one aspect, the DNA-based molecular beacons described herein are introduced into cells, such as live cells, using methods known to those of skill in the art. Such methods include electroporation. In general, foreign nucleic acids (i.e. those which are not part of a cell's natural nucleic acid composition which includes molecular beacons) may be introduced into a cell using any method known to those skilled in the art for such introduction. Such methods include transfection, transduction, viral transduction, microinjection, lipofection, nucleofection, nanoparticle bombardment, transformation, conjugation and the like. One of skill in the art will readily understand and adapt such methods using readily identifiable literature sources.

Exemplary representative molecular beacon sequences for human genes include:

SOX9 (cartilage):
CCGGTCTTTTGGGGGTGGTGGGTGACCGG
PPARG (fat):
CCGGTCAGTGGGAGTGGTCTTCCATTGACCGG
RUNX2 (bone):
CCGGTCCCTGTTGTGTTGTTTGGTAAGACCGG
BCL11B (nerve):
CCGGTCCTTCTATGCTGTTTTTTGTTTGACCGG
IFNG (immunity/inflammation):
CCGGTCTGGTCATCTTTAAAGTTTTTGACCGG
MITF (melanocyte):
CCGGTCAGGGTTAGTATGGATTTCTTGACCGG
MAGEA3/6/12 (multi-gene melanoma):
CCGGTCTCTCTCAAAACCCACTCATGGACCGG,
and
MMP1 (matrix degradation):
CCGGTCTTGTGCGCATGTAGAATCTGGACCGG.

“Complementary” or “substantially complementary” refers to the hybridization or base pairing or the formation of a duplex between nucleotides or nucleic acids, such as, for instance, between the two strands of a double stranded DNA molecule or between an oligonucleotide primer and a primer binding site on a single stranded nucleic acid. Complementary nucleotides are, generally, A and T (or A and U), or C and G. Two single-stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See Kanehisa (1984) Nucl. Acids Res. 12:203.

“Duplex” refers to at least two oligonucleotides and/or polynucleotides that are fully or partially complementary undergo Watson-Crick type base pairing among all or most of their nucleotides so that a stable complex is formed. The terms “annealing” and “hybridization” are used interchangeably to mean the formation of a stable duplex. In one aspect, stable duplex means that a duplex structure is not destroyed by a stringent wash, e.g., conditions including temperature of about 5° C. less that the Tₘ of a strand of the duplex and low monovalent salt concentration, e.g., less than 0.2 M, or less than 0.1 M. “Perfectly matched” in reference to a duplex means that the polynucleotide or oligonucleotide strands making up the duplex form a double stranded structure with one another such that every nucleotide in each strand undergoes Watson-Crick base pairing with a nucleotide in the other strand. The term “duplex” comprehends the pairing of nucleoside analogs, such as deoxyinosine, nucleosides with 2-aminopurine bases, PNAs, and the like, that may be employed. A “mismatch” in a duplex between two oligonucleotides or polynucleotides means that a pair of nucleotides in the duplex fails to undergo Watson-Crick bonding.

“Hybridization” refers to the process in which two single-stranded polynucleotides bind non-covalently to form a stable double-stranded polynucleotide. The term “hybridization” may also refer to triple-stranded hybridization. The resulting (usually) double-stranded polynucleotide is a “hybrid” or “duplex.” “Hybridization conditions” will typically include salt concentrations of less than about 1 M, more usually less than about 500 mM and even more usually less than about 200 mM. Hybridization temperatures can be as low as 5° C., but are typically greater than 22° C., more typically greater than about 30° C., and often in excess of about 37° C. Hybridizations are usually performed under stringent conditions, i.e., conditions under which a probe will hybridize to its target subsequence. Stringent conditions are sequence-dependent and are different in different circumstances. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone. Generally, stringent conditions are selected to be about 5° C. lower than the Tₘ for the specific sequence at a defined ionic strength and pH. Exemplary stringent conditions include salt concentration of at least 0.01 M to no more than 1 M Na ion concentration (or other salts) at a pH 7.0 to 8.3 and a temperature of at least 25° C. For example, conditions of 5×SSPE (750 mM NaCl, 50 mM Na phosphate, 5 mM EDTA, pH 7.4) and a temperature of 25-30° C. are suitable for allele-specific probe hybridizations. For stringent conditions, see for example, Sambrook, Fritsche and Maniatis, Molecular Cloning A Laboratory Manual, 2nd Ed. Cold Spring Harbor Press (1989) and Anderson Nucleic Acid Hybridization, 1^{st} Ed., BIOS Scientific Publishers Limited (1999). “Hybridizing specifically to” or “specifically hybridizing to” or like expressions refer to the binding, duplexing, or hybridizing of a molecule substantially to or only to a particular nucleotide sequence or sequences under stringent conditions when that sequence is present in a complex mixture (e.g., total cellular) DNA or RNA.

“Nucleoside” as used herein includes the natural nucleosides, including 2′-deoxy and 2′-hydroxyl forms, e.g. as described in Komberg and Baker, DNA Replication, 2nd Ed. (Freeman, San Francisco, 1992). “Nucleotide” refers to a phosphorylated nucleoside. “Analogs” in reference to nucleosides includes synthetic nucleosides having modified base moieties and/or modified sugar moieties, e.g., described by Scheit, Nucleotide Analogs (John Wiley, New York, 1980); Uhlman and Peyman, Chemical Reviews, 90:543-584 (1990), or the like, with the proviso that they are capable of specific hybridization. Such analogs include synthetic nucleosides designed to enhance binding properties, reduce complexity, increase specificity, and the like. Polynucleotides comprising analogs with enhanced hybridization or nuclease resistance properties are described in Uhlman and Peyman (cited above); Crooke et al., Exp. Opin. Ther. Patents, 6: 855-870 (1996); Mesmaeker et al., Current Opinion in Structural Biology, 5:343-355 (1995); and the like. Exemplary types of polynucleotides that are capable of enhancing duplex stability include oligonucleotide phosphoramidates (referred to herein as “amidates”), peptide nucleic acids (referred to herein as “PNAs”), oligo-2′-O-alkylribonucleotides, polynucleotides containing C-5 propynylpyrimidines, locked nucleic acids (LNAs), and like compounds. Such oligonucleotides are either available commercially or may be synthesized using methods described in the literature.

“Oligonucleotide” or “polynucleotide,” which are used synonymously, means a linear polymer of natural or modified nucleosidic monomers linked by phosphodiester bonds or analogs thereof. The term “oligonucleotide” usually refers to a shorter polymer, e.g., comprising from about 3 to about 100 monomers, and the term “polynucleotide” usually refers to longer polymers, e.g., comprising from about 100 monomers to many thousands of monomers, e.g., 10,000 monomers, or more. Oligonucleotides usually have lengths in the range of from 12 to 60 nucleotides, and more usually, from 18 to 40 nucleotides. Oligonucleotides and polynucleotides may be natural or synthetic. Oligonucleotides and polynucleotides include deoxyribonucleosides, ribonucleosides, and non-natural analogs thereof, such as anomeric forms thereof, peptide nucleic acids (PNAs), and the like, provided that they are capable of specifically binding to a target genome by way of a regular pattern of monomer-to-monomer interactions, such as Watson-Crick type of base pairing, base stacking, Hoogsteen or reverse Hoogsteen types of base pairing, or the like.

Usually nucleosidic monomers are linked by phosphodiester bonds. Whenever an oligonucleotide is represented by a sequence of letters, such as “ATGCCTG,” it will be understood that the nucleotides are in 5′ to 3′ order from left to right and that “A” denotes deoxyadenosine, “C” denotes deoxycytidine, “G” denotes deoxyguanosine, “T” denotes deoxythymidine, and “U” denotes the ribonucleoside, uridine, unless otherwise noted. Usually oligonucleotides comprise the four natural deoxynucleotides; however, they may also comprise ribonucleosides or non-natural nucleotide analogs. It is clear to those skilled in the art when oligonucleotides having natural or non-natural nucleotides may be employed in methods and processes described herein. For example, where processing by an enzyme is called for, usually oligonucleotides consisting solely of natural nucleotides are required. Likewise, where an enzyme has specific oligonucleotide or polynucleotide substrate requirements for activity, e.g., single stranded DNA, RNA/DNA duplex, or the like, then selection of appropriate composition for the oligonucleotide or polynucleotide substrates is well within the knowledge of one of ordinary skill, especially with guidance from treatises, such as Sambrook et al., Molecular Cloning, Second Edition (Cold Spring Harbor Laboratory, New York, 1989), and like references. Oligonucleotides and polynucleotides may be single stranded or double stranded.

“Specific” or “specificity” in reference to the binding of one molecule to another molecule, such as a target sequence to a probe, means the recognition, contact, and formation of a stable complex between the two molecules, together with substantially less recognition, contact, or complex formation of that molecule with other molecules. In one aspect, “specific” in reference to the binding of a first molecule to a second molecule means that to the extent the first molecule recognizes and forms a complex with another molecule in a reaction or sample, it forms the largest number of the complexes with the second molecule. In certain aspects, this largest number is at least fifty percent. Generally, molecules involved in a specific binding event have areas on their surfaces or in cavities giving rise to specific recognition between the molecules binding to each other. Examples of specific binding include antibody-antigen interactions, enzyme-substrate interactions, formation of duplexes or triplexes among polynucleotides and/or oligonucleotides, receptor-ligand interactions, and the like. As used herein, “contact” in reference to specificity or specific binding means two molecules are close enough that weak non-covalent chemical interactions, such as van der Waal forces, hydrogen bonding, base-stacking interactions, ionic and hydrophobic interactions, and the like, dominate the interaction of the molecules.

“Spectrally resolvable” in reference to a plurality of fluorescent labels means that the fluorescent emission bands of the labels are sufficiently distinct, i.e., sufficiently non-overlapping, that molecular tags to which the respective labels are attached can be distinguished on the basis of the fluorescent signal generated by the respective labels by standard photodetection systems, e.g., employing a system of band pass filters and photomultiplier tubes, or the like, as exemplified by the systems described in U.S. Pat. Nos. 4,230,558; 4,811,218, or the like, or in Wheeless et al., pgs. 21-76, in Flow Cytometry: Instrumentation and Data Analysis (Academic Press, New York, 1985). In one aspect, spectrally resolvable organic dyes, such as fluorescein, rhodamine, and the like, means that wavelength emission maxima are spaced at least 20 nm apart, and in another aspect, at least 40 nm apart. In another aspect, chelated lanthanide compounds, quantum dots, and the like, spectrally resolvable means that wavelength emission maxima are spaced at least 10 nm apart, and in a further aspect, at least 15 nm apart.

“Tₘ” is used in reference to “melting temperature.” Melting temperature is the temperature at which a population of double-stranded nucleic acid molecules becomes half dissociated into single strands. Several equations for calculating the Tₘ of nucleic acids are well known in the art. As indicated by standard references, a simple estimate of the Tₘ value may be calculated by the equation. Tₘ=81.5+0.41 (% G+C), when a nucleic acid is in aqueous solution at 1 M NaCl (see e.g., Anderson and Young, “Quantitative Filter Hybridization,” in Nucleic Acid Hybridization (1985). Other references (e.g., Allawi, H. T. & Santa Lucia, J., Jr., Biochemistry 36, 10581-94 (1997)) include alternative methods of computation which take structural and environmental, as well as sequence characteristics into account for the calculation of Tₘ.

In certain exemplary embodiments, methods for amplifying nucleic acid sequences are provided. Exemplary methods for amplifying nucleic acids include the polymerase chain reaction (PCR) (see, e.g., Mullis et al. (1986) Cold Spring Harb. Symp. Quant. Biol. 51 Pt 1:263 and Cleary et al. (2004) Nature Methods 1:241; and U.S. Pat. Nos. 4,683,195 and 4,683,202), anchor PCR, RACE PCR, ligation chain reaction (LCR) (see, e.g., Landegran et al. (1988) Science 241:1077-1080; and Nakazawa et al. (1994) Proc. Natl. Acad. Sci. U.S.A. 91:360-364), self sustained sequence replication (Guatelli et al. (1990) Proc. Natl. Acad. Sci. U.S.A. 87:1874), transcriptional amplification system (Kwoh et al. (1989) Proc. Natl. Acad. Sci. U.S.A. 86:1173), Q-Beta Replicase (Lizardi et al. (1988) BioTechnology 6:1197), recursive PCR (Jaffe et al. (2000) J. Biol. Chem. 275:2619; and Williams et al. (2002) J. Biol. Chem. 277:7790), the amplification methods described in U.S. Pat. Nos. 6,391,544, 6,365,375, 6,294,323, 6,261,797, 6,124,090 and 5,612,199, isothermal amplification (e.g., rolling circle amplification (RCA), hyperbranched rolling circle amplification (HRCA), strand displacement amplification (SDA), helicase-dependent amplification (HDA), PWGA) or any other nucleic acid amplification method using techniques well known to those of skill in the art.

“Polymerase chain reaction,” or “PCR,” refers to a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors well-known to those of ordinary skill in the art, e.g., exemplified by the references: McPherson et al., editors, PCR: A Practical Approach and PCR2: A Practical Approach (IRL Press, Oxford, 1991 and 1995, respectively). For example, in a conventional PCR using Taq DNA polymerase, a double stranded target nucleic acid may be denatured at a temperature greater than 90° C., primers annealed at a temperature in the range 50-75° C., and primers extended at a temperature in the range 72-78° C. The term “PCR” encompasses derivative forms of the reaction, including but not limited to, RT-PCR, real-time PCR, nested PCR, quantitative PCR, multiplexed PCR, assembly PCR and the like. Reaction volumes range from a few hundred nanoliters, e.g., 200 nL, to a few hundred microliters, e.g., 200 microliters. “Reverse transcription PCR,” or “RT-PCR,” means a PCR that is preceded by a reverse transcription reaction that converts a target RNA to a complementary single stranded DNA, which is then amplified, e.g., Tecott et al., U.S. Pat. No. 5,168,038. “Real-time PCR” means a PCR for which the amount of reaction product, i.e., amplicon, is monitored as the reaction proceeds. There are many forms of real-time PCR that differ mainly in the detection chemistries used for monitoring the reaction product, e.g., Gelfand et al., U.S. Pat. No. 5,210,015 (“Taqman”); Wittwer et al., U.S. Pat. Nos. 6,174,670 and 6,569,627 (intercalating dyes); Tyagi et al., U.S. Pat. No. 5,925,517 (molecular beacons). Detection chemistries for real-time PCR are reviewed in Mackay et al., Nucleic Acids Research, 30:1292-1305 (2002). “Nested PCR” means a two-stage PCR wherein the amplicon of a first PCR becomes the sample for a second PCR using a new set of primers, at least one of which binds to an interior location of the first amplicon. As used herein, “initial primers” in reference to a nested amplification reaction mean the primers used to generate a first amplicon, and “secondary primers” mean the one or more primers used to generate a second, or nested, amplicon. “Multiplexed PCR” means a PCR wherein multiple target sequences (or a single target sequence and one or more reference sequences) are simultaneously carried out in the same reaction mixture, e.g. Bernard et al. (1999) Anal. Biochem., 273:221-228 (two-color real-time PCR). Usually, distinct sets of primers are employed for each sequence being amplified. “Quantitative PCR” means a PCR designed to measure the abundance of one or more specific target sequences in a sample or specimen. Techniques for quantitative PCR are well-known to those of ordinary skill in the art, as exemplified in the following references: Freeman et al., Biotechniques, 26:112-126 (1999); Becker-Andre et al., Nucleic Acids Research, 17:9437-9447 (1989); Zimmerman et al., Biotechniques, 21:268-279 (1996); Diviacco et al., Gene, 122:3013-3020 (1992); Becker-Andre et al., Nucleic Acids Research, 17:9437-9446 (1989); and the like.

It is to be understood that the embodiments of the present invention which have been described are merely illustrative of some of the applications of the principles of the present invention. Numerous modifications may be made by those skilled in the art based upon the teachings presented herein without departing from the true spirit and scope of the invention. The contents of all references, patents and published patent applications cited throughout this application are hereby incorporated by reference in their entirety for all purposes.

The following examples are set forth as being representative of the present invention. These examples are not to be construed as limiting the scope of the invention as these and other equivalent embodiments will be apparent in view of the present disclosure, figures tables and accompanying claims.

### Example I

### Live Cell Temporal Gene Expression Analysis of Osteogenic Differentiation in Adipose-Derived Stem Cells

Cell Culture:

All cells were maintained in a humidified incubator at 37° C. and 5% CO₂. MG-63 and HEK-293 cell lines (ATCC, Manassas, Va.) were cultured in growth medium containing phenol red-free MEM (CellGro, Manassas, Va.) supplemented with 10% FBS, 1% penicillin/streptomycin, 100 mM Glutamax, and 100 mM sodium pyruvate (ThermoFisher Scientific, Waltham, Mass.). Cells were passaged at 80% confluence using 0.25% trypsin-EDTA (ThermoFisher Scientific). For molecular beacon experiments, cells were seeded into 96-well plates at a density of approximately 25,000 cells/cm².

ASCs (adipose-derived stem cells) derived from subcutaneous adipose tissue, originally harvested from seven, healthy, non-diabetic donors between the ages of 18 and 60 years old, were purchased from Zen-Bio, Inc. (superlot #36, Research Triangle Park, N.C.). Cells were grown in expansion medium containing DMEM/F-12 (ThermoFisher Scientific), 10% FBS (Zen-Bio), 1% penicillin/streptomycin, 0.25 ng/mL transforming growth factor-β1, 5 ng/mL epidermal growth factor, and 1 ng/mL fibroblast growth factor (R&D Systems, Minneapolis, Minn.). See Estes B T, Diekman B O, Guilak F. Monolayer cell expansion conditions affect the chondrogenic potential of adipose-derived stem cells. Biotechnology and bioengineering, 99:986-95 (2008) hereby incorproated by reference in its entirety. All ASCs used for experiments were at passage 4.

Beacon Development and Design:

Three custom-designed beacons were developed corresponding to alkaline phosphatase, type I collagen, and osteocalcin mRNA (ALPL, COL1A1 and BGLAP, respectively), which are common markers of osteogenesis. See Table 1 below listing molecular beacon sequences for osteogenic genes. Stem regions are underlined. The remainder of the oligonucleotide forms the loop region, which is complementary to the gene of interest.

TABLE 1

| Gene | Beacon Sequence 5′→3′ |
|---|---|
| GAPDH | CGACGGGAGTCCTTCCACGATACCACGTCG |
| ALPL | CGCTCCAGAGTGTCTTCCGAGGAGGTCAAGGAGCG |
| COL1A1 | CGTCCCAAAAAAAAAAAAAAAAAGAAAAATATCAGGGAGG |
| BGLAP | TCCGCCGGAAAGAAGGGTGCCTGGAGAGGAGCGGCGGA |

Each beacon was functionalized with a 6-FAM (Ex: 492 nm/Em: 517 nm) fluorophore on the 3′ end and a Black Hole Quencher-1 on the 5′ end. A nucleic acid folding program, mfold, was used to model the secondary structures of each mRNA molecule based on thermodynamic stability. See Mathews D H, Turner D H, Zuker M. RNA secondary structure prediction, Current protocols in nucleic acid chemistry/edited by Serge L Beaucage [et al], Chapter 11: Unit 11 2 (2007) and Zuker M., Mfold web server for nucleic acid folding and hybridization prediction, Nucleic Acids Res., 31:3406-15 (2003) each of which are hereby incorproated by reference in their entireties.

The five structures with the lowest Gibbs' free energy were analyzed for regions of largely unpaired or looped secondary structure. A 20-30 base sequence was chosen and assessed using NCBI BLAST to ensure uniqueness. See Mount D W, Using the Basic Local Alignment Search Tool (BLAST), CSH protocols. 2007:pdb top17 (2007) and Altschul S F, Gish W, Miller W, Myers E W, Lipman D J, Basic local alignment search tool, Journal of Molecular Biology, 215:403-10 (1990) each of which are hereby incorporated by reference in their entireties. ALPL, COL1A1, and BGLAP beacons were highly specific to their target sequences (e-values 10⁵, 10², and 10⁶ times smaller than the next sequence match, respectively). The stem region of each beacon was designed to give the probe an optimal melting temperature of 60-80° C. See Bao G, Rhee W J, Tsourkas A., Fluorescent probes for live-cell RNA detection, Annual Review of Biomedical Engineering, 11:25-47 (2009) hereby incorporated by reference in its entirety. The folding of the beacon sequence was also assessed to ensure that a hairpin structure existed. All beacons were manufactured and HPLC purified via commercial sources (MWG Operon, Huntsville, Ala.).

Molecular Beacon Hybridization Assay:

Validation of hybridization efficiency was done by measuring the fluorescence of fixed concentrations of beacon hybridization to varying concentrations of target sequence. Molecular beacon hybridization efficiency was evaluated by exposing a 5 μM concentration of ALPL beacon to stepwise increases in target sequence.

ALPL molecular beacon in pH 7.4 1× Tris-EDTA buffer (ThermoFisher Scientific, 100 μM solution) was added to wells in an opaque 96-well plate at a final beacon concentration of 5 μM/well. Stepwise concentrations of ALPL target sequence (0.5-5.0 μM) were then added to the wells. Controls included wells containing only beacon and Tris buffer, only target and Tris buffer, and only Tris buffer. Sample plates were incubated at 37° C. for 10 minutes, and fluorescence was read with a spectrofluorometer (Spectramax Plus 384, Molecular Devices, Sunnyvale, Calif. Ex: 492 nm, Em: 517 nm) every 10 minutes for a total of 270 minutes. See Bratu D P, Catrina I E, Marras S A., Tiny molecular beacons for in vivo mRNA detection, Methods Mol. Biol. 714:141-57 (2011) hereby incorporated by reference in its entirety.

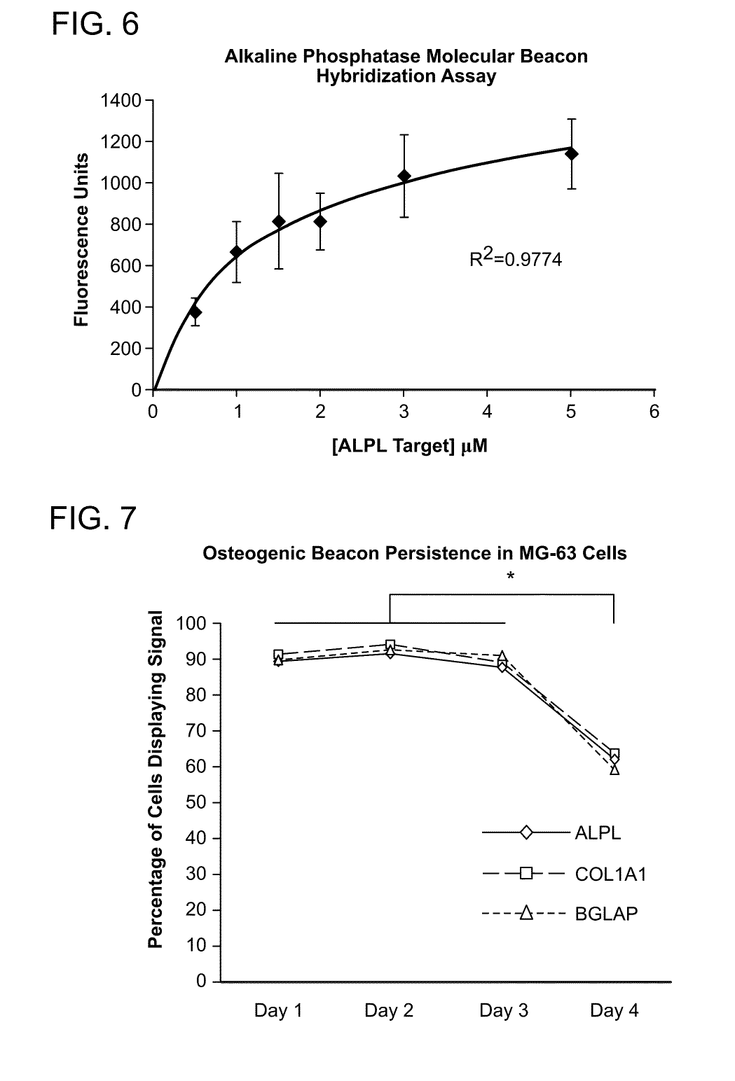

As shown in FIG. 6, at lower concentrations, the fluorescence intensity increased rapidly, while at the highest concentration the binding was saturated (R²=0.98). Average fluorescence values increased three-fold over a target concentration range from 0.5 μM to 5 μM. Beacon fluorescence increased steadily with target concentration, indicating that the beacon readily unfolds and binds to the target. The data suggest that at target concentrations higher than 5 μM, a saturation of binding would occur. These concentrations are ˜1×10⁸ times higher than intracellular concentrations of upregulated mRNA. A signal-to-noise ratio of 24:1 was calculated based on these measurements, which is consistent with previous studies using molecular beacons. See Vet J A, Marras S A., Design and optimization of molecular beacon real-time polymerase chain reaction assays, Methods Mol. Biol., 288:273-90 (2005) hereby incorporated by reference in its entirety.

Beacon Validation and Testing:

MG-63 cells, which highly express osteogenic genes, see Rothem D E, Rothem L, Dahan A, Eliakim R, Soudry M., Nicotinic modulation of gene expression in osteoblast cells, MG-63, Bone, 48:903-9 (2011) hereby incorporated by reference in its entirety, and HEK-293 cells were seeded at a density of 50,000-60,000 cells per well in a 24-well plate. 2 ng of ALPL, COL1A1 and BGLAP molecular beacon (2 μL of 100 μM solution in Tris-EDTA buffer, pH 7.4) were each encapsulated in 4 μL xtremeGENE HP reagent (1:2 ratio beacon:reagent, Roche Biotech, Pleasanton, Calif.) and suspended in 100 μL base medium (MEM) according to product instructions. The complexes were delivered to wells at a concentration of 0.5 μM to ensure that the molecular beacon would be in great excess of the mRNA transcripts (˜6×10¹²-10×10¹² beacons/well), see Mueller S M, Glowacki J., Age-related decline in the osteogenic potential of human bone marrow cells cultured in three-dimensional collagen sponges, Journal of Cellular Biochemistry, 82:583-90 (2001) hereby incorporated by reference in its entirety. A previously published molecular beacon for the housekeeping gene glyceraldehyde-3-phosphate dehydrogenase (GAPDH) was encapsulated and delivered in the same way for use as a positive control. See Santangelo P J, Nitin N, Bao G., Direct visualization of mRNA colocalization with mitochondria in living cells using molecular beacons, Journal of Biomedical Optics, 10:44025 (2005) hereby incorporated by reference in its entirety. Following beacon treatment, cells were allowed an uptake period of at least 2 hours before being imaged on a Nikon Eclipse Ti—U epifluorescent microscope (Nikon Instruments Inc., Melville, N.Y.). Images were captured with a scope-mounted QICAM 12-bit digital camera (Qimaging, Surrey, BC, Canada). Signal intensity for presented figures was uniformly thresholded to minimize background levels.

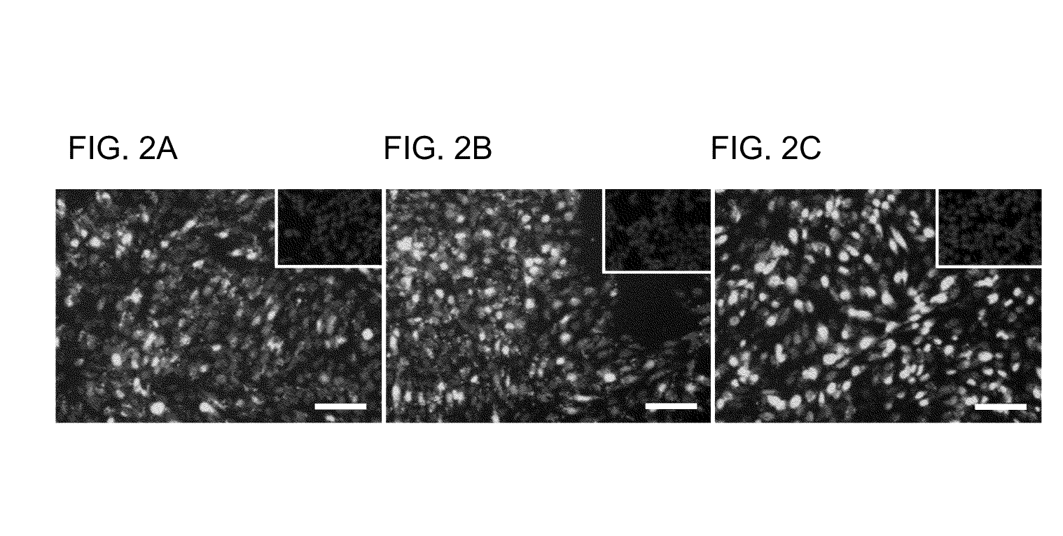

As shown in FIG. 2, Molecular beacons for ALPL (A), COL1A1 (B), and BGLAP (C) were tested in MG-63 (positive control) and HEK-293 (negative control, insets) cells to ensure functionality and specificity. Fluorescence signals were observed throughout the MG-63 cells, while no signals were observed in HEK-293 cells. Color enhanced for presentation purposes. Scale bars: 100 μm. After administration of GAPDH beacon to MG-63 and HEK-293 cells, 97-99% of cells in each sample population displayed positive signal. MG-63 and HEK-293 cells were treated with molecular beacons corresponding to ALPL, COL1A1, and BGLAP. 95-99% of treated MG-63 cells showed robust positive signal for all three beacons. In treated HEK-293 cells, only 1-2% of cells showed positive signal. All cells displayed typical morphology and remained spread over a 72-hour observation period. To validate the expression levels of osteogenic genes detected by molecular beacons, RT-qPCR was performed to quantify the mRNA levels of COL1A1 and BGLAP in beacon-treated MG-63 and HEK-293 cells. Both mRNA levels were greatly increased in MG-63 cells versus HEK-293 cells.

RT-qPCR Gene Expression Verification:

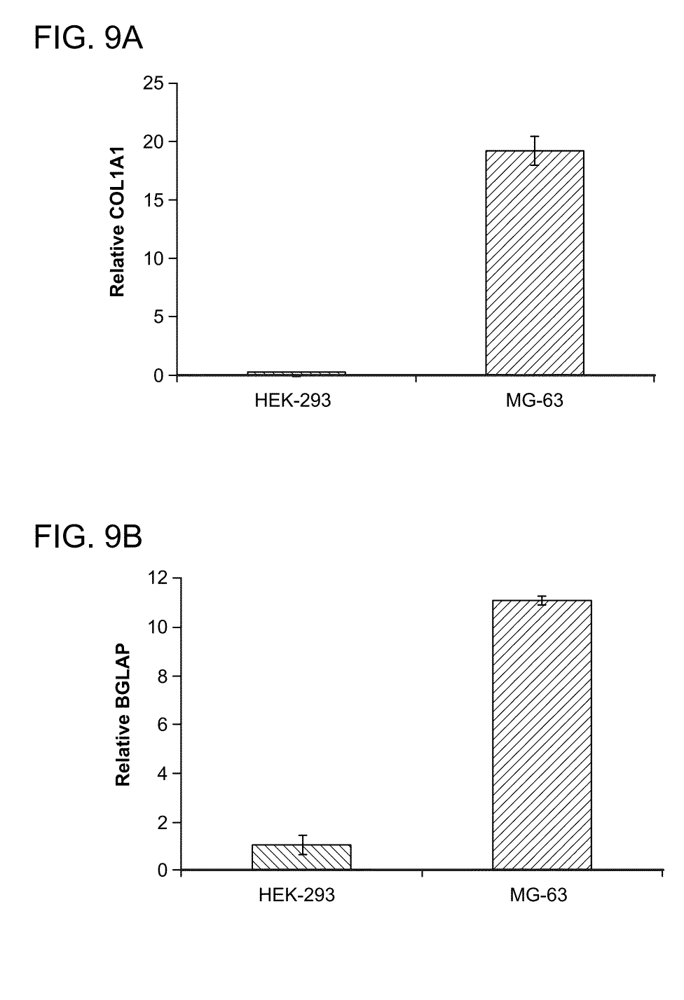

mRNA was isolated from MG-63 cells and HEK-293 cells using the RNAqueous Kit (Ambion, Austin, Tex.) according to manufacturer's instructions. Real-time quantitative polymerase chain reaction (RT-qPCR) was conducted using the DNA Engine Opticon 2 (Bio-Rad, Hercules, Calif.) using the QuantiTect SYBR Green PCR kit (Qiagen, Valencia, Calif.). For RT-qPCR, 0.5 ug of RNA was reverse-transcribed using iScript cDNA Synthesis Kit (Bio-Rad) according to the manufacturer's instructions. Species-specific primer pairs were used to compare COL1A1 and BGLAP mRNA transcript levels in MG-63 and HEK-293 cells. Calculations were done using the delta delta Ct (AACt) method, normalized to rRNA 18S expression. RT-qPCR data were collected for COL1A1 and BGLAP in MG-63 cells to verify that high gene expression levels corresponded with positive beacon signals. MG-63 cells exhibited significantly greater mRNA copy numbers for the osteogenic genes COL1A1 (FIG. 9A) and BGLAP (FIG. 9B) compared to HEK-293 cells (p<0.001). The elevated COL1A1 and BGLAP mRNA levels, as measured by RT-qPCR, corroborated molecular beacon data showing that an overwhelmingly higher percentage of MG-63 cells positively expressed these osteogenic genes compared to HEK-293 cells.

For discussion of RT-qPcR to evaluate ASC osteogenesis, see Frank 0, Heim M, Jakob M, Barbero A, Schafer D, Bendik I, et al., Real-time quantitative RT-PCR analysis of human bone marrow stromal cells during osteogenic differentiation in vitro, Journal of Cellular Biochemistry, 85:737-46 (2002); Pittenger M F, Mackay A M, Beck S C, Jaiswal R K, Douglas R, Mosca J D, et al., Multilineage potential of adult human mesenchymal stem cells, Science, 284:143-7 (1999) and Zuk P A, Zhu M, Ashjian P, De Ugarte D A, Huang J I, Mizuno H, et al., Human adipose tissue is a source of multipotent stem cells, Molecular Biology of the Cell, 13:4279-95 (2002) each of which are hereby incorporated by reference in its entirety.

Osteogenic Differentiation of ASCs:

ASCs were chemically induced for osteogenesis following established protocols. See Bunnell B A, Estes B T, Guilak F, Gimble J M., Differentiation of adipose stem cells, Methods Mol. Biol., 456:155-71 (2008) hereby incorporated by reference in its entirety. Control medium contained DMEM/F-12 (ThermoFisher Scientific), 10% FBS (Zen-Bio), and 1% antibiotic/antimycotic. Osteogenic induction medium included the addition of 1 nM dexamethasone, 21.6 mg/mL β-glycerophosphate, 50 μg/mL ascorbate-2-phosphate, and 10 μg/mL vitamin D3 (Sigma-Aldrich, St. Louis, Mo.). Two separate, 96-well plates were seeded with 30,000 cells/well in 24 wells per plate using control medium. All cells were treated with 1 ng/mL Hoechst dye to visualize nuclei (Sigma-Aldrich). After 24 hours, 12 wells were given 180 μL osteogenic medium while the remaining 12 wells were given 180 μL control medium. 90% of the medium was changed every other day for twenty-one days.

Beacon Treatment and Imaging of Differentiating ASCs:

mRNA-specific beacons were introduced to cells during the twenty-one day differentiation process. From Days 2-10, four osteogenic wells and four control wells were treated with ALPL beacon as described above and imaged daily. For Days 8-16, four separate osteogenic and control wells were treated with COL1A1 beacon and imaged daily. For Days 17-21, the remaining four osteogenic and control wells were treated with BGLAP beacon and imaged daily. Four fields of view in each well were taken of Hoechst-stained nuclei, fluorescent beacon signals, and bright field images of cells at 10× magnification (sixteen fields of view total for osteogenic/control conditions). Cells were treated with the appropriate beacons on Days 2, 5, 7, 10, 14 and 17 to maintain saturating intracellular concentrations. This re-treatment schedule was chosen based on a beacon persistence assay in living cells that indicated that the signal was diminished by Day 4. In particular and as shown in FIG. 7, MG-63 osteosarcoma cells were treated with beacons corresponding to ALPL, COL1A1, and BGLAP and monitored daily for four days to assess persistence of the DNA molecular beacons in live cells. Beacon signal remained consistent for Days 1-3 but decreased by >30% between Days 3 and 4 (p<0.05).

Image Processing and Analysis:

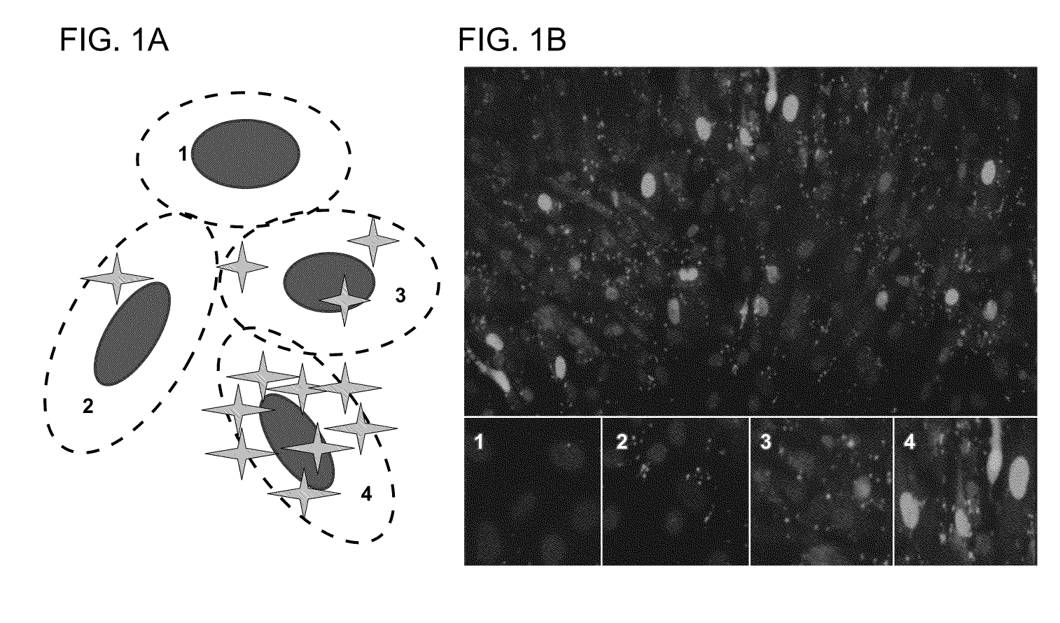

CellProfiler image analysis software was used to generate a MATLAB-based algorithm that relates “child” fluorescent signals to “parent” Hoechst-stained nuclei. See Lamprecht M R, Sabatini D M, Carpenter A E., CellProfiler: free, versatile software for automated biological image analysis, BioTechniques, 42:71-5 (2007) and Carpenter A E, Jones T R, Lamprecht M R, Clarke C, Kang I H, Friman O, et al., CellProfiler: image analysis software for identifying and quantifying cell phenotypes, Genome Biology, 7:R100 (2006) each of which are hereby incorporated by reference in their entireties. This relation of fluorescence signal to parent nuclei is valid since no extracellular fluorescence was observed in any of the images. The software set thresholding parameters for each image by first identifying the maximum and minimum values of pixel intensity in each image, then defining all pixel intensities in the lowest 20% as background. The program analyzed each set of images by first counting Hoescht-stained nuclei, which were recognized as ellipsoidal objects with a major axis between 5 and 20 microns (10 and 40 pixels). An area encompassing the nuclear/perinuclear region (25×10 μm ellipsoid) was defined when monitoring fluorescent signal in each cell. Individual fluorescence events were identified by pixel and grouped with the nearest nucleus, thus defining a cell as displaying positive signal or not. Accordingly, the percentage of cells with positive signals for specific genes of interest was calculated using image analysis algorithms. A cartoon representation of the analysis is shown in FIG. 1A illustrating the basic concept. In FIG. 1B, fluorescence signals were assigned to the nearest Hoescht-stained nuclei, establishing parent-child relationships between the two images (FIG. 1B, merged for illustration purposes). “Low/no-signal” cells exhibited no fluorescence events in the region of interest above a minimum threshold value (A1, B1). “Positive” signals varied in type and intensity and included point signals (A2, B2), punctate, compressed speckling (A3, B3), and widespread fluorescence throughout the perinuclear region (A4, B4). All categories were included when calculating percentage of positive cells.

The number of total fluorescing cells was divided by the total number of cells per image, giving a percentage of positively signaling cells for each sample well. Possible sources of error for this method included classifying weak signals as background, attributing signals to the incorrect cell due to overlapping nuclei, and discarding signals that were outside the analysis area. Despite these sources, however, the error rate from this method was only ±8%, which is comparable to human error for similar samples. This analysis relied heavily on a preexisting modification to the CellProfiler program offered by the Broad Institute; the program and its modifications can be viewed at world wide website cellprofiler.org and world wide website cellprofiler.org/CPmanual/RelateObjects.html.

Verification of Osteogenesis:

Alkaline phosphatase activity in differentiating ASCs was determined according to instructions for the BioVision alkaline phosphatase assay kit (Mountain View, Calif.). Briefly, 4 induced and 4 control wells per plate were either treated with ALPL molecular beacon or left untreated. After seven days, these cells were lysed in 200 μL lysis buffer. Lysate was stored at −80° C. until testing. For analysis, lysates were thawed and centrifuged at 13,000 rpm for 5 minutes. 50 μL of the resulting solutions were transferred into individual wells of a 96-well plate and brought to volume with 110 μL of lysis buffer. The remaining 50 μL from each sample were transferred into separate wells on the same plate, brought to volume, and then treated with 20 μL stop solution to act as background controls. A standard curve using 0-0.5 mM alkaline phosphatase was made for quantification of samples. All wells were treated with 10 μL of 5 mM methylumbelliferone-4-phosphate solution for detection. The wells were covered and incubated at room temperature for 30 minutes, after which stop solution was added to all wells. A spectrofluorometer (Spectramax Plus 384, Molecular Devices) determined the fluorescence of each well at 360 nm/440 nm.

Alizarin Red-S (ARS, Sigma-Aldrich) staining was done for both control and osteogenically induced wells to examine calcified matrix production. ARS (2% in distilled water) was pH-adjusted (4.1-4.3) and filtered through a 0.2 μm pore filter prior to use. On Day 21, beacon-treated wells were fixed with 3.7% paraformaldehyde in PBS (ThermoFisher Scientific). The fixed cell monolayers were washed in distilled water for 5 minutes, stained with 2% ARS for 20 minutes, and then thoroughly rinsed. After staining was complete, wells were imaged at 20× magnification using bright field microscopy and a scope-mounted digital camera (Labomed TCM 400, Labomed, Culver City, Calif.). ARS dye was then eluted with 10% cetylpyridinium chloride (ThermoFisher Scientific) overnight at 4° C., and the optical densities were measured at 540 nm with a spectrofluorometer (Spectramax Plus 384, Molecular Devices).

Statistical Analysis:

Gene expression patterns were determined using percent expression in differentiating ASC populations (n=4 for ALPL, COL1A1, and BGLAP). Data were analyzed using two-factor ANOVA (treatment, time; α=0.05) with Fisher's LSD post-hoc analysis. Osteogenic protein depositions were analyzed using a Student's T-test for control (n=4) and induced (n=4) samples. False positive and negative results of 1-2% were sufficiently small and had a minimal impact on the interpretation of results.

### Example II

### Molecular Beacon Signaling in Differentiating ASCs

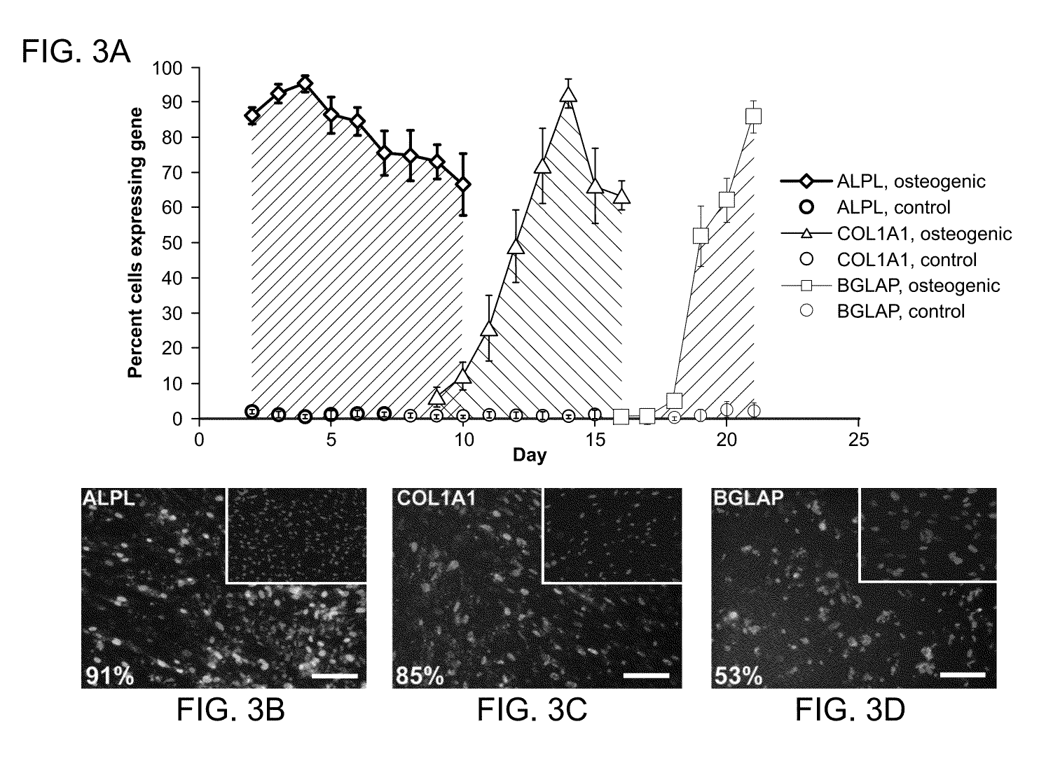

ASCs undergoing osteogenic differentiation were treated with molecular beacons for ALPL, COL1A1, and BGLAP in a stepwise fashion over twenty-one days. Percentage of positively signaling cells were calculated to monitor temporal gene expression patterns. FIG. 3 is a graph of the percentage of cells expressing ALPL, COL1A1, and BGLAP measured daily from Days 2-21. Expression percentages reflect the expected gene expression profiles for a differentiating population of ASCs (filled symbols). Peak percentages represent the point at which most cells in the induced population displayed positive signal for the gene of interest. These values can also be used as a measure of stem cell purity since only positively differentiating cells should express all three osteogenic genes. Control populations (open symbols) have signal levels close to zero, indicating a lack of osteogenic gene expression. ALPL beacon-treated ASCs showed consistent positive signals during the first week of differentiation, starting at 86% on Day 2, peaking at 92-95% on Days 3 and 4 (p<0.0001), and decreasing to 63% on Day 10. COL1A1 beacon-treated ASCs showed 6% positively signaling cells at Day 9, rising to a peak of 91% on Day 14 (p<0.0001), and dropping off to 64% by Day 16. Less than 1% of the ASC population showed positive BGLAP beacon signal from Days 16-18, but the percentage of cells increased to 52% by Day 19 and reached a peak percentage of 86% on Day 21 (p<0.0001), the final day of testing. Control ASCs that were not induced for osteogenesis showed less than 2% fluorescence in all instances.

### Example III

### Verification of Osteogenesis

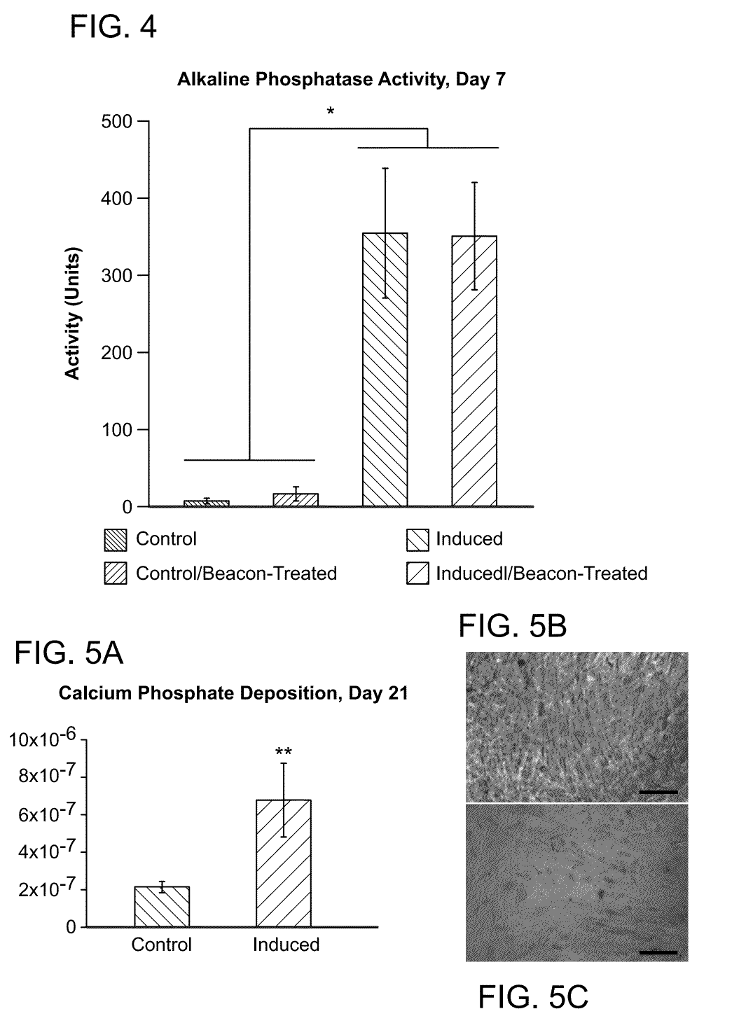

FIG. 4 is a graph showing alkaline phosphatase activity in control and induced ASC populations were significantly different, regardless of beacon presence (*p<0.0001). Activity in control samples was near zero, while activity in induced samples was two orders of magnitude higher. There were no significant differences in activity between beacon-treated and untreated samples undergoing osteogenesis, serving as both a signal of successful differentiation and evidence for uninterrupted protein synthesis in the presence of molecular beacons.

On Day 7 of osteogenesis, both beacon-treated and untreated induced ASC samples had approximately 300 units of alkaline phosphatase activity per cell, while all control ASCs had approximately 5-10 units of activity per cell. The alkaline phosphatase activity assay revealed protein activity two orders of magnitude higher in osteogenic samples than for controls, in both beacon-treated and untreated samples. There were no significant differences in activity between beacon-treated and untreated samples undergoing osteogenesis, serving as both a signal of successful differentiation and evidence for uninterrupted protein synthesis in the presence of molecular beacons, i.e. beacon hybridization to mRNA did not interfere with protein synthesis.

Chemically induced ASCs successfully underwent osteogenesis over 21 days. FIG. 5A is a graph of absorbance values for eluted Alizarin Red-S dye which indicated that induced samples deposited three times more calcified matrix than control samples (p<0.0001). Induced (FIG. 5B) and control (FIG. 5C) ASCs stained with Alizarin Red-S for 30 minutes showed clear, qualitative differences in calcified matrix deposition. Scale bars: 100 μm. On Day 21, the optical density of Alizarin Red dye eluted from osteogenically induced cells was three times higher than in control samples (FIG. 5A, p<0.0001). Imaging of stained cells revealed a bright crimson color in osteogenically induced samples, while control samples retained little of the dye (FIG. 5B-C). Alizarin red staining for calcified matrix is a commonly used and well-established method for assessing osteogenic differentiation. Staining with alizarin red and testing for activity with the alkaline phosphatase assay were meant to ensure that differentiation had indeed occurred successfully, which would not have been possible had significant gene knockdown occurred.

### Example IV

### Discussion

Adipose-derived stem cells, like other mesenchymal stem cells, display considerable heterogeneity in their ability to differentiate in a uniform fashion. The Examples above assessed gene expression patterns during osteogenic differentiation at the subpopulation and population levels, thereby investigating heterogeneity in differentiating adipose-derived stem cell cultures. Differentiating ASC populations displayed clear, dynamic expression patterns of osteogenic genes, assessed by measuring the percentage of positively signaling cells over a three week period.

The examples above in established cell lines, MG-63 and HEK-293, and using a housekeeping gene, GAPDH, verify specificity and uptake of the molecular beacon into >97% of the cells in a population. Subsequent measurement of the percentage of fluorescent, and therefore expressing, cells in the differentiating ASC populations showed a distinct pattern of upregulation followed by a slow decline in the number of expressing cells. Initial expression levels began lower for all genes (less than 10% for COL1A1 and BGLAP) and then slowly rose to a peak of greater than 90% in all cases. Thus, peak expression times of characteristic genes in osteogenesis were identified. In the Examples above, the percentage of positive signal cells increased in larger jumps during the later phases of osteogenesis. The ERK/Akt pathways have been implicated in osteogenic lineage commitment and have previously been induced by culture in type I collagen-coated flasks, implying that the presence of type I collagen in the extracellular environment may assist in committing a cell to the osteogenic lineage. See Tsai K S, Kao S Y, Wang C Y, Wang Y J, Wang J P, Hung S C., Type I collagen promotes proliferation and osteogenesis of human mesenchymal stem cells via activation of ERK and Akt pathways, Journal of Biomedical Materials Research Part A, 94:673-82 (2010) hereby incorporated by reference in its entirety. The data for ALPL gene expression showed data points very close together in their slow rise and fall from the peak percentage point, with a difference of only a few percentage points between Days 3 and 4. Mapping patterns for COL1A1 and BGLAP showed that the percentages jump from around 50% to 75% and from 60% to about 90%, respectively, over a single day. From these patterns, it can be inferred that the cells lock into osteogenesis after an initial induction period during which type I collagen is upregulated. While differentiation begins slowly in the first week, it quickly picks up speed and momentum, with more and more cells joining the differentiation process and settling into a more uniform gene expression pattern in later weeks.

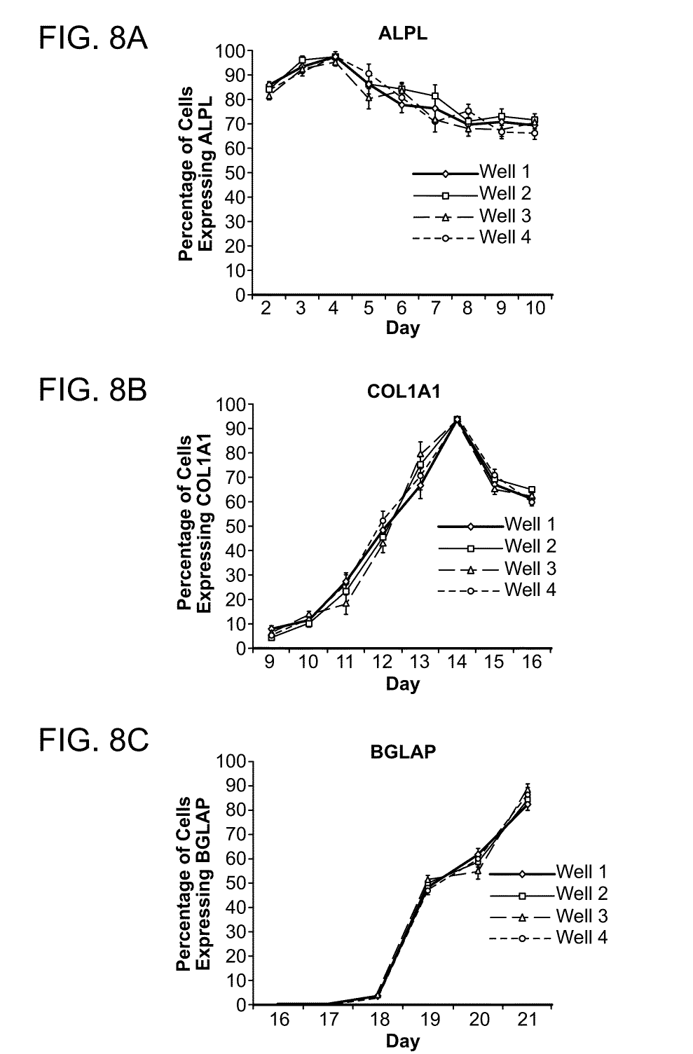

Notably, each cell appears to be on its own track during differentiation. Some cells express the genes of interest very early on; for example, around 85% of cells express ALPL on Day 2, though the peak percentage of cells expressing ALPL occurs on Day 4. Cells that express genes early may also stop expressing genes sooner, which would explain the gradual decrease from the peak percentage as opposed to a steep drop. Additionally, when expression percentages are analyzed on a per-well basis, the changes in expression patterns from well-to-well are most apparent for ALPL, begin to even out for COL1A1, and are almost non-existent for BGLAP (FIGS. 8A, 8B and 8C showing the variation in percentage of cells that displayed positive signals for each mRNA target on a well-by-well basis). FIGS. 8A, 8B and 8C show differences among the wells (e.g., ALPL, Day 5 and 7.) These differences lessen as differentiation proceeds (e.g., BGLAP shows uniform expressions on almost all days.) These expression patterns serve as additional evidence for the heterogeneity of stem cell populations, given the apparent differences in variation in gene expression from well to well during osteogenesis. Additionally, it is important to note the possibility that even when nearly every cell from a population is differentiating, not all of them so do in the same timeframe.

### Example V

### Isolating Lineage-Specific Cells from a Population of Cells

Aspects of the present disclosure are directed to identifying and/or isolating lineage-specific cells, i.e. cells which have been induced to under differentiation, from a population of cells where a subpopulation may not have been induced to undergo differentiation. In this manner, a population of cells can be created which is enriched for cells undergoing differentiation. According to this aspect, molecular beacons are hybridized to mRNA markers and the cells with the molecular beacons hybridized to the mRNA can then be isolated or separated from the population of cells based on the label. For example, hybridization of molecule beacon having a fluorescent label can result in activation of the fluorescent label. The cells with the activated fluorescent label can then be sorted, such as by using FACS (fluorescence activated cell sorting.) Exemplary procedures and provided below.

Molecular Beacon Design:

A custom-designed beacon was developed corresponding to alkaline phosphatase mRNA (ALPL), which is a marker of osteogenesis. See Tyagi S, Kramer FR., Molecular beacons: Probes that fluoresce upon hybridization. Nature Biotechnology. 1996; 14(3):303-8, Epub 1996/03/01, doi: 10.1038/nbt0396-303, PubMed PMID: 9630890, and Desai H V, Voruganti I S, Jayasuriya C, Chen Q, Darling E M., Live-cell, temporal gene expression analysis of osteogenic differentiation in adipose-derived stem cells. Tissue Engineering Part A, 2013; 19(1-2):40-8, Epub 2012/07/31, doi: 10.1089/ten.TEA.2012.0127., PubMed PMID: 22840182; PubMed Central PMCID: PMC3530940 each of which are hereby incorporated by reference in their entireties.

Each beacon was functionalized with a 6-FAM (Ex: 492 nm/Em: 517 nm) fluorophore on the 3′ end and a Black Hole Quencher-1 on the 5′ end. A nucleic acid folding program, mfold, was used to model the secondary structures of each mRNA molecule based on thermodynamic stability. See Zuker M., Mfold web server for nucleic acid folding and hybridization prediction, Nucleic Acids Research, 2003; 31(13):3406-15, Epub 2003/06/26, PubMed PMID: 12824337; PubMed Central PMCID: PMC169194 hereby incorporated by reference in its entirety. The five structures with the lowest Gibbs' free energy were analyzed for regions of largely unpaired or looped secondary structure. A 20-30 base sequence was chosen and assessed using NCBI BLAST to ensure uniqueness. See Altschul S F, Gish W, Miller W, Myers E W, Lipman D J., Basic local alignment search tool, Journal of Molecular Biology, 1990; 215(3):403-10, Epub 1990/10/05, doi: 10.1016/S0022-2836(05)80360-2, PubMed PMID: 2231712 hereby incorporated by reference in its entirety. The stem region of each beacon was designed to give the probe an optimal melting temperature of 60-80° C. See Bao G, Rhee W J, Tsourkas A., Fluorescent probes for live-cell RNA detection, Annual Review of Biomedical Engineering, 2009; 11:25-47, Epub 2009/04/30, doi: 10.1146/annurev-bioeng-061008-124920, PubMed PMID: 19400712; PubMed Central PMCID: PMC2734976 hereby incorporated by reference in its entirety. The folding of the beacon sequence was also assessed to ensure that a hairpin structure existed. All beacons were manufactured and HPLC purified via commercial sources (MWG Operon, Huntsville, Ala.). The sequence of the beacon is as follows (stems underlined): 5′ (6-Carboxyfluorescein) CGCTCC AGAGTGTCTTCCGAGGAGGTCAA GGAGCG (Black Hole Quencher 1) 3′.

Adipose-Derived Stromal Cell Isolation:

Adipose-derived stromal cells (ASCs) were isolated from the subcutaneous adipose tissue of three human, female donors following established protocols. See Estes B T, Diekman B O, Gimble J M, Guilak F., Isolation of adipose-derived stem cells and their induction to a chondrogenic phenotype, Nature Protocols, 2010; 5(7):1294-311, Epub 2010/07/03, doi: 10.1038/nprot.2010.81, PubMed PMID: 20595958; PubMed Central PMCID: PMC3219531 hereby incorporated by reference in its entirety. Briefly, the liposuction waste tissue was washed with warm phosphate buffered saline at pH 7.4 and then digested with lmg/mL collagenase solution for 60 minutes. The released cells were washed four times with stromal medium (Dulbecco's Modified Eagle's Medium in a 1:1 ratio with Ham's F12 salt solution, 10% fetal bovine serum, and 1% antibiotic/antimycotic). The cells were cryogenically stored at a concentration of 5-6×10⁶ cells/mL. For designated preliminary/pilot studies, an ASC “superlot” containing 7 non-diabetic donors between the ages of 18 and 60 was purchased commercially (Zen-Bio, Research Triangle Park, N.C.).

Medium Compositions:

Stromal medium contained Dulbecco's Modified Eagle's Medium in a 1:1 ratio with Ham's F12 salt solution, 10% fetal bovine serum, and 1% antibiotic/antimycotic. Expansion medium had the same basic composition as stromal medium, with the addition of 5 ng/mL epidermal growth factor, 1 ng/mL fibroblast growth factor, and 0.25 ng/mL transforming growth factor β1 to maintain the cells' undifferentiated state. See Estes BT, Diekman B O, Guilak F., Monolayer cell expansion conditions affect the chondrogenic potential of adipose-derived stem cells, Biotechnology and Bioengineering, 2008; 99(4):986-95, Epub 2007/10/12, doi: 10.1002/bit.21662, PubMed PMID: 17929321 hereby incorporated by reference in its entirety. Osteogenic induction medium (OIM) contained Dulbecco's Modified Eagle's Medium with high glucose, 10% fetal bovine serum, 1% antibiotic/antimycotic, 1 nM dexamethasone, 21.6 mg/mL β-glycerophosphate, 50 μg/mL ascorbate-2-phosphate, and 10 μg/mL vitamin D3 (Sigma-Aldrich). See Gonzalez-Cruz R D, Fonseca V C, Darling E M., Cellular mechanical properties reflect the differentiation potential of adipose-derived mesenchymal stem cells, Proceedings of the National Academy of Sciences of the United States of America, 2012; 109(24):E1523-9, Epub 2012/05/23, doi: 10.1073/pnas.1120349109, PubMed PMID: 22615348; PubMed Central PMCID: PMC3386052 hereby incorporated by reference in its entirety. Adipogenic induction medium (AIM) contained DMEM/F-12, 3% FBS, 1% antibiotic/antimycotic, 10 μg/mL insulin, 0.39 μg/mL dexamethasone, 55.6 μg/mL isobutyl-1-methylxanthine (Sigma-Aldrich), and 17.5 μg/mL indomethacin (Cayman Chemical). Chondrogenic induction medium (CIM) contained Dulbecco's Modified Eagle's Medium with high glucose, 10% fetal bovine serum, 1% antibiotic/antimycotic, 10 ng/mL TGF-β1, 50 μg/mL ascorbate-2-phosphate, 39.0 ng/mL dexamethasone, and 1% ITS+Premix (BD Biosciences).

Osteogenic Induction and Molecular Beacon Treatment:

Freshly thawed, primary ASCs were seeded in monolayer at 33,000 cells/cm² and given osteogenic induction medium (OIM). A separate flask of the same cells was kept in expansion medium to maintain the cells' undifferentiated state as a control. Four days after induction, both induced and non-induced cells were trypsinized using 0.25% trypsin-EDTA and resuspended in 100 μL non-supplemented base medium (DMEM:F12) at a concentration of 10×10⁶ cells/mL. ALPL molecular beacon was added to the resulting cell suspension to bring beacon concentration to 1 μM. The prescribed amount of cell and molecular beacon suspension was added to electroporation cuvettes. For cuvettes with a 0.2 cm gap, 200 μL of cell suspension was added per cuvette. For cuvettes with a 0.4 cm gap, 400 μL of cell suspension was added. The electroporator was set according to manufacturer's instructions at program U-23 for voltage and pulse length, and cells were electroporated. When the process was complete, the cuvette was removed and sequentially rinsed with 500 μL of stromal medium three times to collect all cells in a total of 1.5 mL medium. The cells were allowed to rest for 60 minutes in a 37° C. incubator with 5% CO₂. Cells were then centrifuged at 400 g for 5 minutes to pellet cells and resuspended at a concentration of 10×10⁶ cells/mL for fluorescence activated cell sorting (FACS) in 37° C. unsupplemented, optically clear buffer. Samples were protected from light until sorting was begun. All sorts were initiated within 1 hour of electroporation.

Fluorescence Activated Cell Sorting:

All sorts were performed on a BD FACS Aria IIc instrument. Cell samples (induced and non-induced) treated with ALPL beacon were sorted following standard protocols. The instrument was outfitted with an extra-wide 100 μm nozzle to minimize pressure during the sorting procedure. The forward scatter (FSC) threshold was set at 5000 units. Cells were sorted by FACS into positive (ALPL+) and negative (ALPL−) populations. A subset of cells from the initial, beacon-treated population was left unsorted as a control.

Cell Seeding and Differentiation:

Following FACS, ALPL+, ALPL−, and unsorted cells were seeded in 96-well plates at 25,000 cells/well and differentiated down the osteogenic and adipogenic lineages using the induction media described previously. An equivalent number of ALPL+, ALPL−, and unsorted cells from the sort were seeded in the same fashion and maintained in stromal medium as a control. For chondrogenic differentiation, 50,000 cells/well from each group (ALPL+, ALPL−, unsorted) were seeded in a V-bottomed 96-well plate and centrifuged at 400 g to form cell pellets conducive to chondrogenesis. The cells were then given chondrogenic induction media to induce chondrogenesis or stromal medium to act as a control.

Staining and Quantification of Adipogenesis and Osteogenesis:

After 14 days, adipogenesis samples were fixed with 10% formalin buffered saline and stained with Oil Red 0, a dye that binds intracellular lipids indicative of adipogenesis. The Oil Red 0 dye was eluted from fixed cells using 100% isopropanol, and the absorbance (A) of the eluents was measured at 500 nm. After 21 days, osteogenesis samples were fixed and stained with Alizarin Red-S, which binds to calcified matrix and is indicative of bone formation. After staining, the dye was eluted using 10% cetylpyridinium phosphate, and the absorbance of the eluent was measured at 540 nm. Absorbance measurements were normalized on a per cell basis by staining nuclei with DAPI and quantifying images using CellProfiler cell counting software (A/20,000 cells). See Carpenter A E, Jones T R, Lamprecht M R, Clarke C, Kang I H, Friman O, Guertin D A, Chang J H, Lindquist R A, Moffat J, Golland P, Sabatini D M., CellProfiler: image analysis software for identifying and quantifying cell phenotypes, Genome Biology, 2006; 7(10):R100, Epub 2006/11/02, doi: 10.1186/gb-2006-7-10-r100, PubMed PMID: 17076895; PubMed Central PMCID: PMC1794559 hereby incorporated by reference in its entirety.

Assessment of Chondrogenesis:

After 21 days, chondrogenically induced and non-induced pellets were digested with 125 μg/mL pH 6.5 papain at 65° C. for 24 hours (Sigma-Aldrich). The sGAG content of each digested pellet was quantified using the dimethylmethylene blue (DMMB) assay, modified from established protocols. See Guilak F, Lott K E, Awad H A, Cao Q, Hicok K C, Fermor B, Gimble J M., Clonal analysis of the differentiation potential of human adipose-derived adult stem cells, Journal of Cellular Physiology, 2006; 206(1):229-37, Epub 2005/07/16, doi: 10.1002/jcp.20463, PubMed PMID: 16021633 and Awad H A, Halvorsen Y D, Gimble J M, Guilak F., Effects of transforming growth factor beta1 and dexamethasone on the growth and chondrogenic differentiation of adipose-derived stromal cells, Tissue Engineering, 2003; 9(6):1301-12, Epub 2003/12/13, doi: 10.1089/10763270360728215, PubMed PMID: 14670117 each of which are hereby incorporated by reference in its entirety. Briefly, 2.1 mg DMMB was dissolved in 100% ethanol, then combined with a solution of 304 mg glycine and 237 mg sodium chloride in 3 M HCl. The pH of the DMMB dye solution was adjusted to 1.5 using 6 M HCl. 200 μL of this prepared dye solution was added to 50 μL of digest solution in wells of a 96-well plate. The optical densities of the resulting mixtures were measured at 525 nm. The PicoGreen assay (Invitrogen) was used to quantify DNA amounts using 100 μL of each digest (480 nm excitation, 520 nm emission). A standard curve was used to calculate total sGAG amounts in each pellet, which were then normalized on a per-DNA basis.

### Example VI

### Discussion

ALPL molecular beacon-based sorting of adipose-derived stromal cells was readily accomplished using FACS and yielded clear populations of positive and negative cells. Roughly half of the sorted cells from non-expanded ASC samples displayed ALPL mRNA expression versus typical yields of around a third when CD34+, CD31− cells were sorted using surface markers. More stringent FACS-based sorting schemes for mesenchymal stem cells have resulted in yields much less than 1%. See Gronthos S, Zannettino A C., A method to isolate and purify human bone marrow stromal stem cells, Methods Mol. Biol., 2008; 449:45-57, Epub 2008/03/29, doi: 10.1007/978-1-60327-169-1_{—}3, PubMed PMID: 18370082 hereby incorporated by reference in its entirety. ALPL+ cells had a strong propensity for osteogenesis, showing a 550% increase in osteogenic matrix deposition compared to unsorted ASCs (p=4.71×10-5). The ALPL+ cells also showed a 200% increase in lipid formation, indicative of adipogenesis, and a 170% increase in sulfated glycosaminoglycan production, indicative of chondrogenesis, over unsorted cells. These results indicated that molecular beacon-based sorting using ALPL dramatically increases osteogenic matrix production as a result of lineage-specific enrichment of ASCs for osteogenesis. Furthermore, ALPL beacon-based sorting yielded multipotent subpopulations of cells in high yield compared to traditional surface marker-based sorting. See Bourin P, Bunnell B A, Casteilla L, Dominici M, Katz A J, March K L, Redl H, Rubin J P, Yoshimura K, Gimble J M., Stromal cells from the adipose tissue-derived stromal vascular fraction and culture expanded adipose tissue-derived stromal/stem cells: a joint statement of the International Federation for Adipose Therapeutics and Science (IFATS) and the International Society for Cellular Therapy (ISCT), Cytotherapy, 2013; 15(6):641-8, Epub 2013/04/11, doi: 10.1016/j.jcyt.2013.02.006. PubMed PMID: 23570660 hereby incorporated by reference in its entirety.

Sort Data and Cell Yield:

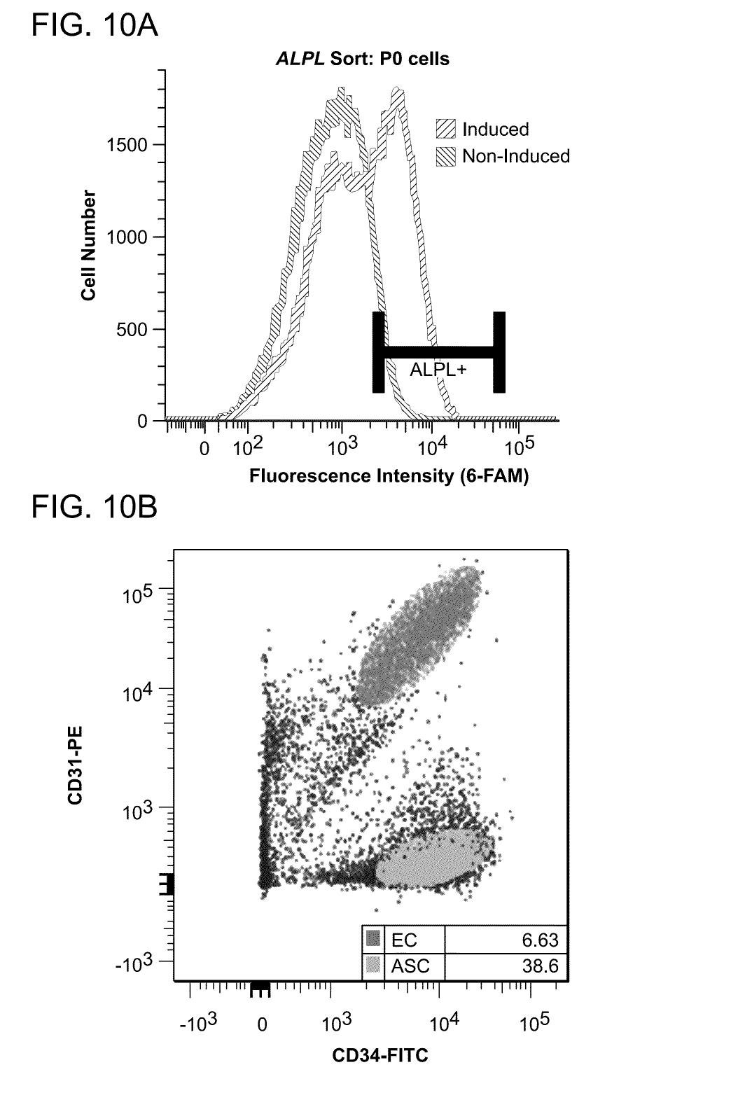

ASC sort data showed that approximately 48% of cells sorted displayed positive beacon signal (ALPL+) while approximately 46% of ASCs were classified as low/no signal (ALPL−, see FIG. 10A). This is an increase in yield over traditional surface marker based sorting, where ASCs defined as CD34+/CD31− cells were only 38% of the assessed population (see FIG. 10B).

Osteogenic Differentiation:

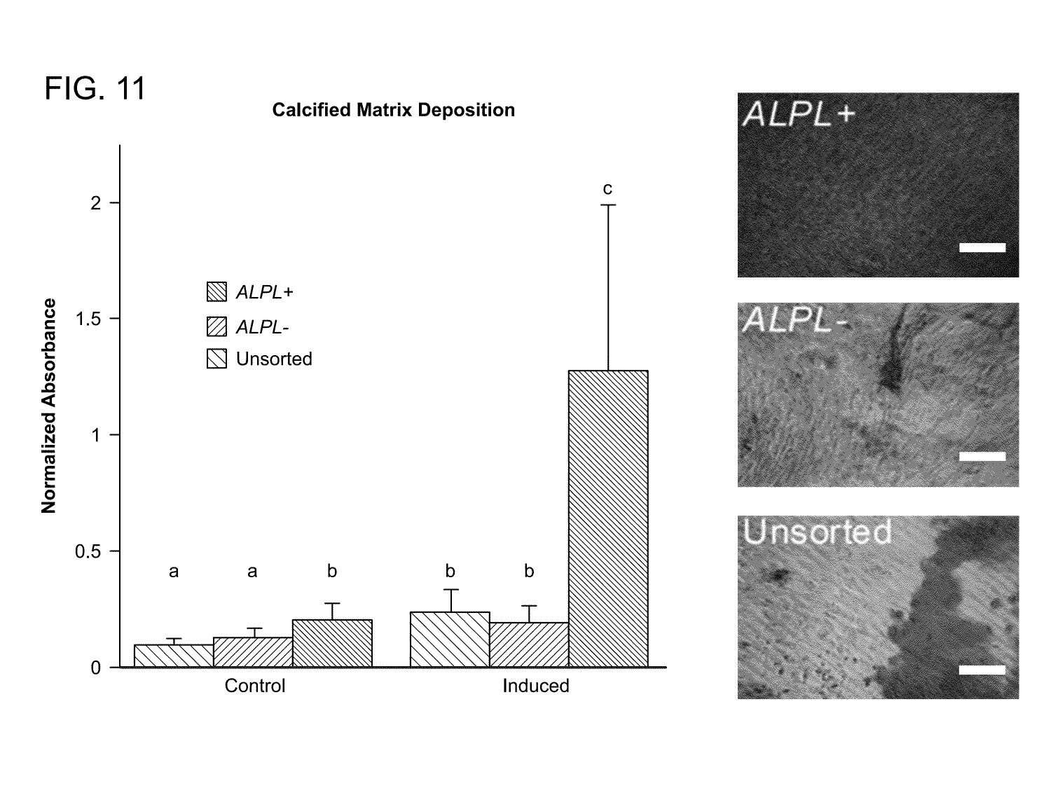

Greatly enhanced osteogenic differentiation existed in ALPL+ cells compared to both unsorted and ALPL− populations, resulting in a significant increase in the deposition of calcified matrix (see FIG. 11). Remarkably, ALPL+ cells displayed a 5.5-fold increase in calcified matrix deposition over unsorted ASCs (A: 1.27±0.71 vs. 0.22±0.10, p<0.05) and a 6.7-fold increase over ALPL− cells (A: 0.19±0.06, p<0.05). While unsorted cells did differentiate, the quality and extent of matrix formation was inferior to that of the ALPL+ cells (1.3-fold increase versus 5.4-fold increase, respectively, between induced and control samples). Also interesting to note is that even within control populations, ALPL+ cells were able to produce significantly more matrix than unsorted and ALPL− control cells (p=1.7×10-4).

Adipogenic Differentiation:

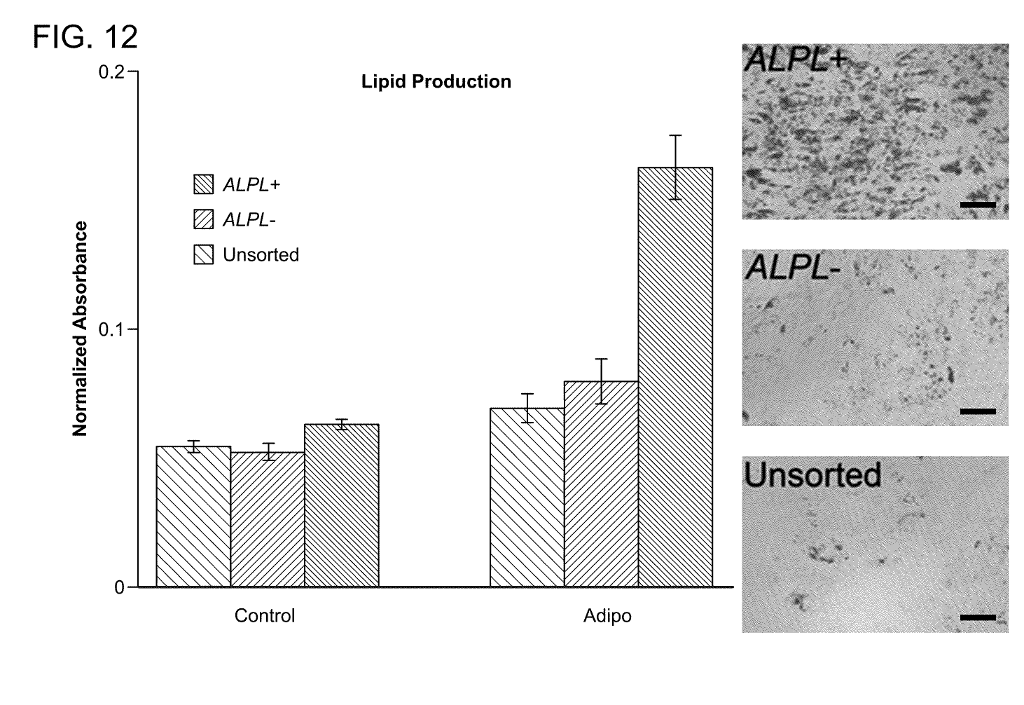

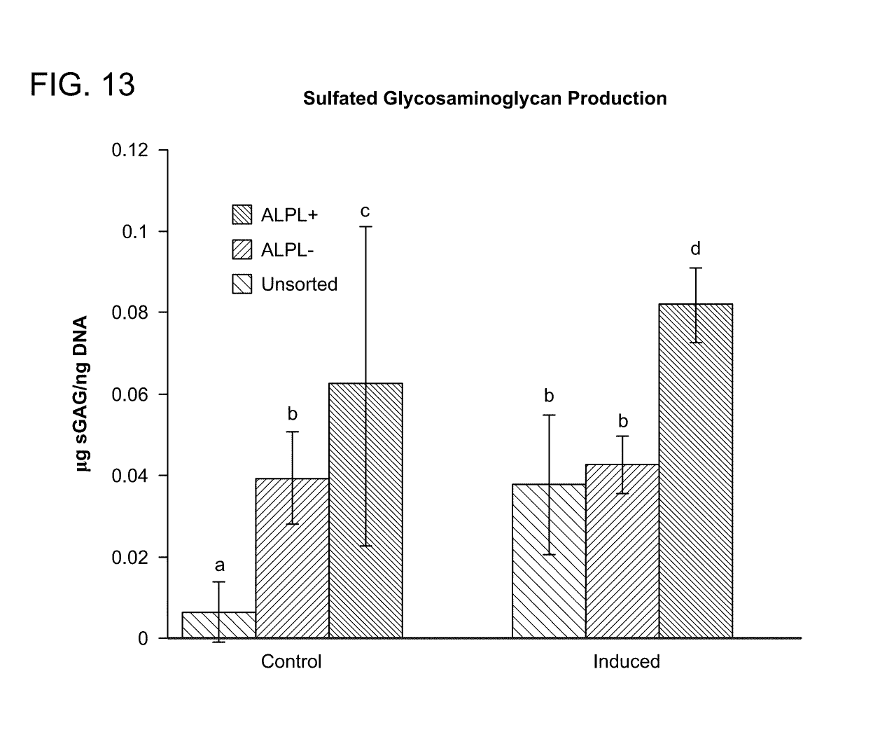

To determine whether ALPL sorting identified a unipotent or multipotent phenotype, additional lineages beyond osteogenesis were assessed. ALPL+ cells were the only group to differentiate down the adipogenic lineage, as evidenced by the 1.6-fold increase in lipid production in induced cells versus control cells (see FIG. 12. A: 0.16±0.01 vs. 0.06±0.002, p<0.05). ALPL− and unsorted cells did not successfully differentiate [A(ALPL−): 0.08±0.008 vs. 0.05±0.003; A(Unsorted): 0.07±0.005 vs. 0.05±0.002]. Additionally, ALPL+ induced cells displayed a 1.4-fold increase in lipid production over unsorted induced cells, and a 1-fold increase in lipid production of ALPL− induced cells. These results imply that cells that would otherwise be unable to differentiate down the adipogenic lineage are able to do so once they have been sorted using ALPL molecular beacons.

Chondrogenic Differentiation:

To further determine whether ALPL sorting displayed a multipotent phenotype, chondrogenesis was also assessed. Chondrogenesis was indicated by production of sulfated glycosaminoglycans (sGAG) normalized by ng DNA per sample. Chondrogenically induced ALPL+ cells produced significantly more sGAG than any other group (see FIG. 13, 0.082 μg sGAG), displaying 1.7- and 0.9-fold increases over unsorted and ALPL− samples, respectively (0.037 μg sGAG and 0.042 μg sGAG, p<0.05). Notably, uninduced ALPL+ cells produced significantly more sGAG than all groups except induced ALPL+ cells (0.062 μg sGAG, p<0.05), which serves as further evidence of ASC enrichment. ALPL− cells were the only group unable to differentiate down the chondrogenic lineage.

### Example VII

### Methods for Designing Molecular Beacons to Hybridize to Target mRNA Markers of Lineage-Specific Cells

According to certain aspects, methods are provided for the design of molecular beacons to hybridize to target mRNA markers of lineage-specific cells, for example within a population of cells. Messenger RNA (mRNA) is upregulated rapidly in response to stimuli, and is more exposed and available for binding than tightly packaged genomic DNA. This makes it an ideal candidate for detection via molecular beacon technology, in which a DNA-based hybridization probe recognizes and binds to mRNA of interest in living cells. The mRNA has a secondary structure that arises from intramolecular hybridization of the 3000-4000 base molecules. This folding is unable to be measured exactly, but algorithms that model the folding of mRNA based on thermodynamic principles can very closely approximate the state of the mRNA in its globular, folded form. To design effective and successful molecular beacons, the present disclosure provides conditions or parameters on which to design molecular beacons. According to one aspect, the conditions and parameters are implemented by software code whereby the target mRNA sequence is inputted into the program and the program determines molecular beacon sequences suitable for binding to the target mRNA marker, such as a target mRNA marker for a lineage-specific cell, based on the conditions and parameters. Using the conditions and parameters, one can generate potential beacon sequences for any mRNA of interest. According to certain aspects, the method includes one or more or all of the following conditions and parameters and steps.

1. Obtain all mRNA sequence isoforms/variants for gene of interest via the National Library of Medicine/PubMed Gene database or other suitable database or other source of information. This can be accomplished by searching the gene name and species of interest in the search utility (e.g. alkaline phosphatase homo sapiens).

2. Model folding of all sequences by calling an external, DNA/RNA folding program (UNAFold). See Zuker M., Mfold web server for nucleic acid folding and hybridization prediction, Nucleic Acids Research, 2003; 31(13):3406-15, Epub 2003/06/26, PubMed PMID: 12824337; PubMed Central PMCID: PMC169194 hereby incorporated by reference in its entirety. Useful information and considerations include all potential folded mRNA structures using thermodynamic assumptions, resulting in ˜20-50 structures per variant, information such as Gibbs free energy and melting temperature of folded mRNA, whether nucleotides are single-stranded or bound and the probability of the nucleotide being in that state.

3. Once the mRNA folding structures have been generated, MATLAB is used to identify highly likely regions for most successful molecular beacon binding/hybridization.

4. The MATLAB code first generates all possible beacons for the target mRNA sequence by simply making nucleotide sequences X nt long, advancing one nucleotide at a time. X can range anywhere from about 10 to about 35 nucleotides long, or more exemplary from about 15 to about 25 nucleotides long. The single-strandedness and probability associated with each nucleotide is used to determine the suitability of each short nucleotide sequence as a potential beacon. The potential beacon sequences are ranked in order of most single-stranded to least, ignoring any sequences that are less than 60% single-stranded, although that can be raised or lowered depending on desired stringency.

5. For potential beacons, a generic “stem” sequence of 5-6 nucleotides is added to either end to allow it to form a hairpin loop. This stem sequence can be shortened/lengthened or shuffled to allow for an optimal melting temperature for the beacon.

6. The external folding program is used to determine if the potential beacon forms the optimal stem-loop structure. If it does, then it is kept for further consideration, otherwise it is removed from the analysis.

7. Potential beacons are then assessed for specificity by calling NCBI's BLAST (Basic Local Alignment Search Tool) program which looks at how similar/complimentary the molecular beacon sequence is to ALL existing mRNA in the organism of interest. See Altschul S F, Gish W, Miller W, Myers E W, Lipman D J., Basic local alignment search tool, Journal of Molecular Biology, 1990; 215(3):403-10, Epub 1990/10/05, doi: 10.1016/S0022-2836(05)80360-2, PubMed PMID: 2231712 hereby incorporated by reference in its entirety.

8. A suitable molecular beacon has an “Expect” or “e-value” less than 1 and the next, non-target mRNA sequence has an e-value greater than 1. These values can be altered as needed to make the selection more or less restrictive.

9. A list of potential beacons is thus generated for each variant of a target mRNA sequence, if there is more than 1 variant. If there is more than 1 variant, all sequences are compared and only their complimentary regions are kept. This can be modified to remove a subset of variants as needed or to only generate beacons for a single variant by targeting only the dissimilar regions of the variant of interest.

10. The MATLAB program outputs a text file containing the location of the sequence on the mRNA molecule, the loop sequence, the entire beacon sequence with stem, a graphical depiction of the beacon, delta G, average single-strandedness, and certainty/probability value. Based on the delta G, the stem sequence can be changed to obtain an optimal melting temperature. Typically, this means a delta G between −3.7 and −2.5 kCal. Alternatively, a low and high melting temperature can be considered which is determined by modeling melting at varying salt and solute concentrations. One of skill will readily understand how to determine optimum melting temperature.

11. The final molecular beacon candidates are assessed by hand as well as BLAST and UNAFold to verify sufficient specificity and appropriate binding locations in the mRNA molecule. The first and last ˜100 nt of the mRNA sequence is often avoided if other good options exist.

Using the above conditions, parameters and steps, molecular beacons were designed for target mRNA markers of several exemplary lineage specific cells including adipogenic cells, neurogenic cells, osteogenic cells, chondrogenic cells and peripheral blood mononuclear cells. Exemplary molecular beacons useful for hybridizing to mRNA markers for adipogenic cells are shown in FIG. 14. Exemplary molecular beacons useful for hybridizing to mRNA markers for neurogenic cells are shown in FIG. 15. Exemplary molecular beacons useful for hybridizing to mRNA markers for osteogenic cells are shown in FIG. 16. Exemplary molecular beacons useful for hybridizing to mRNA markers for chondrogenic cells are shown in FIG. 17. Exemplary molecular beacons useful for hybridizing to mRNA markers for peripheral blood mononuclear cells are shown in FIG. 18.

### Example VIII

### Molecular Beacons to Hybridize to Target mRNA Markers of Cancerous Cells

Aspects of the present disclosure are directed to identifying and/or isolating malignant cells from a population of cells including non-malignant cells. Exemplary malignant cells include cancer cells, however one of skill will readily understand that other malignant cells can be identified and diagnosed based on the methods described herein using molecular beacons to hybridize to target mRNA marker of the cells. In this manner, a method of diagnosing malignancies, such as cancer, is provided. In this manner, a method of diagnosing cancer is provided. According to this aspect, molecular beacons are hybridized to mRNA markers for cancer cells and the cells with the molecular beacons hybridized to the mRNA can then be imaged or isolated or separated from the population of cells based on the label. For example, hybridization of molecule beacon having a fluorescent label can result in activation of the fluorescent label. The cells with the activated fluorescent label can then be imaged or sorted, such as by using FACS (fluorescence activated cell sorting.) Exemplary procedures described herein. Using the methods described herein molecular beacons were designed for target mRNA markers of several exemplary cancer cells including melanoma, squamous cell carcinoma and basal cell carcinoma. Exemplary molecular beacons useful for hybridizing to mRNA markers for melanoma are shown in FIG. 19. Exemplary molecular beacons useful for hybridizing to mRNA markers for squamous cell carcinoma are shown in FIG. 20. Exemplary molecular beacons useful for hybridizing to mRNA markers for basal cell carcinoma are shown in FIG. 21.

According to certain aspects, mRNA markers are known to be associated with various cancers. Based on the mRNA marker, one or more molecular beacons can be designed to hybridize with the mRNA marker for the cancer cell. Exemplary cancers within the scope of the present disclosure include cancers of the breast, skin, bone, prostate, ovaries, uterus, cervix, liver, lung, brain, larynx, gallbladder, pancreas, rectum, parathyroid, thyroid, adrenal gland, immune system, neural tissue, head and neck, colon, stomach, bronchi, and/or kidneys. Examples of general categories of cancer include, but are not limited to, carcinomas (i.e., malignant tumors derived from epithelial cells such as, for example, common forms of breast, prostate, lung and colon cancer), sarcomas (i.e., malignant tumors derived from connective tissue or mesenchymal cells), lymphomas (i.e., malignancies derived from hematopoietic cells), leukemias (i.e., malignancies derived from hematopoietic cells), germ cell tumors (i.e., tumors derived from totipotent cells. In adults most often found in the testicle or ovary; in fetuses, babies and young children, most often found on the body midline, particularly at the tip of the tailbone), blastic tumors (i.e., a typically malignant tumor which resembles an immature or embryonic tissue) and the like.

Examples of specific neoplasms intended to be encompassed by the present invention include, but are not limited to, acute lymphoblastic leukemia; myeloid leukemia, acute myeloid leukemia, childhood; adrenocortical carcinoma; AIDS-related cancers; AIDS-related lymphoma; anal cancer; appendix cancer; astrocytoma (e.g., cerebellar, cerebral); atypical teratoid/rhabdoid tumor; basal cell carcinoma; bile duct cancer, extrahepatic; bladder cancer; bone cancer, osteosarcoma and malignant fibrous histiocytoma; brain tumor (e.g., brain stem glioma, central nervous system atypical teratoid/rhabdoid tumors, central nervous system embryonal tumors, cerebellar astrocytoma, cerebral astrocytoma/malignant glioma, craniopharyngioma, ependymoblastoma, ependymoma, medulloblastoma, medulloepithelioma, pineal parenchymal tumors of intermediate differentiation, supratentorial primitive neuroectodermal tumors and/or pineoblastoma, visual pathway and/or hypothalamic glioma, brain and spinal cord tumors); breast cancer; bronchial tumors; Burkitt lymphoma; carcinoid tumor (e.g., gastrointestinal); carcinoma of unknown primary; central nervous system (e.g., atypical teratoid/rhabdoid tumor, embryonal tumors (e.g., lymphoma, primary); cerebellar astrocytoma; cerebral astrocytoma/malignant glioma; cervical cancer; chordoma; chronic lymphocytic leukemia; chronic myelogenous leukemia; chronic myeloproliferative disorders; colon cancer; colorectal cancer; craniopharyngioma; cutaneous T-cell lymphoma; embryonal tumors, central nervous system; endometrial cancer; ependymoblastoma; ependymoma; esophageal cancer; Ewing family of tumors; extracranial germ cell tumor; extragonadal germ cell tumor; extrahepatic bile duct cancer; eye cancer (e.g., intraocular melanoma, retinoblastoma); gallbladder cancer; gastric cancer; gastrointestinal tumor (e.g., carcinoid tumor, stromal tumor (gist), stromal cell tumor); germ cell tumor (e.g., extracranial, extragonadal, ovarian); gestational trophoblastic tumor; glioma (e.g., brain stem, cerebral astrocytoma); hairy cell leukemia; head and neck cancer; hepatocellular cancer; Hodgkin lymphoma; hypopharyngeal cancer; hypothalamic and visual pathway glioma; intraocular melanoma; islet cell tumors; Kaposi sarcoma; kidney cancer; large cell tumors; laryngeal cancer (e.g., acute lymphoblastic, acute myeloid); leukemia (e.g., acute myeloid, chronic lymphocytic, chronic myelogenous, hairy cell); lip and/or oral cavity cancer; liver cancer; lung cancer (e.g., non-small cell, small cell); lymphoma (e.g., AIDS-related, Burkitt, cutaneous T-cell, Hodgkin, non-Hodgkin, primary central nervous system); macroglobulinemia, Waldenström; malignant fibrous histiocytoma of bone and/or osteosarcoma; medulloblastoma; medulloepithelioma; melanoma; merkel cell carcinoma; mesothelioma; metastatic squamous neck cancer; mouth cancer; multiple endocrine neoplasia syndrome; multiple myeloma/plasma cell neoplasm; mycosis fungoides; myelodysplastic syndromes; myelodysplastic/myeloproliferative diseases; myelogenous leukemia (e.g., chronic, acute, multiple); myeloproliferative disorders, chronic; nasal cavity and/or paranasal sinus cancer; nasopharyngeal cancer; neuroblastoma; non-Hodgkin lymphoma; non-small cell lung cancer; oral cancer; oral cavity cancer, oropharyngeal cancer; osteosarcoma and/or malignant fibrous histiocytoma of bone; ovarian cancer (e.g., ovarian epithelial cancer, ovarian germ cell tumor, ovarian low malignant potential tumor); pancreatic cancer (e.g., islet cell tumors); papillomatosis; paranasal sinus and/or nasal cavity cancer; parathyroid cancer; penile cancer; pharyngeal cancer; pheochromocytoma; pineal parenchymal tumors of intermediate differentiation; pineoblastoma and supratentorial primitive neuroectodermal tumors; pituitary tumor; plasma cell neoplasm/multiple myeloma; pleuropulmonary blastoma; primary central nervous system lymphoma; prostate cancer; rectal cancer; renal cell cancer; renal, pelvis and/or ureter, transitional cell cancer; respiratory tract carcinoma involving the nut gene on chromosome 15; retinoblastoma; rhabdomyosarcoma; salivary gland cancer; sarcoma (e.g., Ewing family of tumors, Kaposi, soft tissue, uterine); Sézary syndrome; skin cancer (e.g., non-melanoma, melanoma, merkel cell); small cell lung cancer; small intestine cancer; soft tissue sarcoma; squamous cell carcinoma; squamous neck cancer with occult primary, metastatic; stomach cancer; supratentorial primitive neuroectodermal tumors; T-cell lymphoma, cutaneous; testicular cancer; throat cancer; thymoma and/or thymic carcinoma; thyroid cancer; transitional cell cancer of the renal, pelvis and/or ureter; trophoblastic tumor; unknown primary site carcinoma; urethral cancer; uterine cancer, endometrial; uterine sarcoma; vaginal cancer; visual pathway and/or hypothalamic glioma; vulvar cancer; Waldenström macroglobulinemia; Wilms tumor and the like. For a review, see the National Cancer Institute's Worldwide Website (cancer.gov/cancertopics/alphalist). One of skill in the art will understand that this list is exemplary only and is not exhaustive, as one of skill in the art will readily be able to identify additional cancers and/or neoplasms based on the disclosure herein.

### EQUIVALENTS

Other embodiments will be evident to those of skill in the art. It should be understood that the foregoing description is provided for clarity only and is merely exemplary. The spirit and scope of the present invention are not limited to the above example, but are encompassed by the claims. All publications, patents and patent applications cited above are incorporated by reference herein in their entirety for all purposes to the same extent as if each individual publication or patent application were specifically indicated to be so incorporated by reference.

1. A method for enriching live tissue lineage-specific cells from a population of live cells comprising
subjecting the population of live cells to conditions inducing differentiation to a specific tissue type, wherein differentiation is induced in a plurality of the live cells to produce live tissue lineage-specific cells expressing a mRNA marker,
hybridizing the mRNA marker within the live tissue lineage-specific cells to a molecular beacon having a label attached thereto, and
isolating the live tissue lineage-specific cells based on the label.

2. The method of claim 1 wherein the live cells include unipotent, bipoptent, multipotent, pluripotent or totipotent cells.

3. The method of claim 1 wherein the live cells include stem cells, embryonic stem cells, pluripotent stem cells, induced pluripotent stem cells, mesenchymal stem cells, somatic adult stem cells, stromal cells or progenitor cells.

4. The method of claim 1 wherein the label is a fluorescent label.

5. The method of claim 1 wherein the label is a fluorescent label and the live tissue lineage-specific cells are isolated based on the fluorescent label.

6. The method of claim 1 wherein the label is a fluorescent label and the live tissue lineage-specific cells are isolated using fluorescence activated cell sorting.

7. The method of claim 1 wherein the label is a magnetic label.

8. The method of claim 1 wherein the label is a magnetic label and the live tissue lineage-specific cells are isolated based on the magnetic label.

9. The method of claim 1 wherein the label is a magnetic label and the live tissue lineage-specific cells are isolated using a magnetic field.

10. A method for identifying live tissue lineage-specific cells within a population of live cells comprising
subjecting the population of live cells to conditions inducing differentiation to a specific tissue type, wherein differentiation is induced in a plurality of the live cells to produce live tissue lineage-specific cells expressing a mRNA marker,
hybridizing the mRNA marker within the live tissue lineage-specific cells to a molecular beacon having a label attached thereto, and
identifying the live tissue lineage-specific cells based on the label.

11. The method of claim 10 wherein the live cells include unipotent, bipoptent, multipotent, pluripotent or totipotent cells.

12. The method of claim 10 wherein the live cells include stem cells, embryonic stem cells, pluripotent stem cells, induced pluripotent stem cells, mesenchymal stem cells, somatic adult stem cells, stromal cells or progenitor cells.

13. The method of claim 10 wherein the label is a fluorescent label.

14. The method of claim 10 wherein the label is a fluorescent label and the live tissue lineage-specific cells are identified based on the fluorescent label.

15. The method of claim 10 wherein the label is a fluorescent label and the live tissue lineage-specific cells are identified using imaging of the fluorescent label.

16. The method of claim 10 wherein the label is a magnetic label.

17. The method of claim 10 wherein the label is a magnetic label and the live tissue lineage-specific cells are identified based on the magnetic label.

18. The method of claim 10 wherein the label is a magnetic label and the live tissue lineage-specific cells are identified using a magnetic field.

19. A method of optimizing production of a specific tissue type from cells induced to differentiate into the specific tissue type comprising
subjecting a population of live cells to conditions inducing differentiation to the specific tissue type, wherein differentiation is induced in a plurality of the live cells to produce live tissue lineage-specific cells expressing a mRNA marker,
hybridizing the mRNA marker within the live tissue lineage-specific cells to a molecular beacon having a label attached thereto,
isolating the live tissue lineage-specific cells based on the label, and
further inducing differentiation of the isolated live tissue lineage-specific cells into the specific tissue type.

20. The method of claim 19 wherein the live cells include unipotent, bipoptent, multipotent, pluripotent or totipotent cells.

21-61. (canceled)
